(19) United States
(12) Patent Application Publication
(10) Pub. No.: US 2019/0300842 A1
(43) Pub. Date: Oct. 3, 2019

(54) SYSTEMS AND METHODS FOR CONTINUOUSLY CULTURING MICROALGAE IN MIXOTROPHIC CONDITIONS

(71) Applicant: HELIAE DEVELOPMENT, LLC, GlLBERT, AZ (US)

(72) Inventors: Eneko GANUZA TABERNA, TEMPE, AZ (US); April COBOS, Gilbert, AZ (US); Rachel MADDOX, Gilbert, AZ (US); Jenna LLOYD-RANDOLFI, Chandler, AZ (US); Mason OELSCHLAGER, Gilbert, AZ (US); Luke CIZEK, Mesa, AZ (US); Kara PAOLILLI-BAUTISTA, Irvine, CA (US)

(73) Assignee: HELIAE DEVELOPMENT, LLC, GILBERT, AZ (US)

(21) Appl. No.: 16/307,775

(22) PCT Filed: Jun. 16, 2017
(86) PCT No.: PCT/US2017/038035

§ 371 Date: Dec. 6, 2018

Related U.S. Application Data
(60) Provisional application No. 62/351,415, filed on Jun. 17, 2016.

(51) Int. Cl. C12N 1/12; C12M 1/00; C12M 1/26; C12M 1/34; C12M 1/36

(52) U.S. Cl. CPC C12N 1/12; C12M 21/02; C12M 33/00; C12M 47/02; C12M 41/48; C12M 41/06; C12M 41/26

(57) ABSTRACT

Methods of culturing microalgae in a continuous auxostat system are described herein. The methods may be used in mixotrophic culture conditions, and may be used in systems comprising open or closed bioreactor systems.

### CROSS-REFERENCE TO RELATED APPLICATIONS

This application claims priority to International Application No. PCT/US2017/038035, filed Jun. 16, 2017, which claims the benefit of U.S. Provisional Application No. 62/351,415, filed Jun. 17, 2017, entitled System and Methods for Continuously Culturing Microalgae in Mixotrophic Conditions, all of which are incorporated by reference herein in their entirety.

### BACKGROUND

Production of microorganisms, such as microalgae and cyanobacteria, can provide feedstock for a variety of products including fuel, nutrition, material, and agricultural products. One advantage microalgae and cyanobacteria provide over traditional terrestrial plant feedstocks for such products is the capability to grow in non-arable lands and produce a unique product profile. While microalgae and cyanobacteria have the potential to complement production of terrestrial plants, the efficiency of current microalgae and cyanobacteria culturing systems and methods need to improve to make the technology economically feasible. Increased efficiency can be provided by growing microalgae mixotrophically. Mixotrophic cultures have been produced in batch and semi-continuous systems, but the changing cell densities observed in those systems do not allow for control of the contribution of each metabolism (i.e., photosynthesis and heterotrophy) to mixotrophic growth, resulting in suboptimal process control and productivities. The inventors describe a method of continuous mixotrophic cultivation for better controlling the cultivation process and improve productivity at large scale.

### SUMMARY

Some embodiments include methods of culturing microalgae in a continuous auxostat system are described for cultures in mixotrophic and heterotrophic conditions. Benefits of the methods comprise: providing means to control the availability of light while maintaining productivity, providing means for selecting for the population of highest producing cells, improving growth, improving productivity, and improving culture longevity. The methods may be implemented with closed and open bioreactors systems. The continuous auxostat methods may also be implemented with batch or semi-continuous culturing methods in a previous or subsequent stage.

### DESCRIPTION OF FIGURES

FIG. 1 shows a graph of the dry weight for a microalgae culture in batch and continuous mode operation.

FIG. 2 shows a graph of the acetic acid and sodium nitrate uptake for a microalgae culture in batch and continuous mode operation.

FIG. 3 shows a graph of the productivity for a microalgae culture in batch and continuous mode operation.

FIG. 4 shows a graph of the dry weight, productivity, and biomass for a microalgae culture in batch and continuous mode operation.

FIG. 5 shows a graph of the productivity for a microalgae culture in batch and continuous mode operation.

FIG. 6 shows a graph of the acetic acid, nitrogen, and phosphate concentration for a microalgae culture in batch and continuous mode operation.

FIG. 7 shows a graph of the dry weight and dissolved oxygen for a microalgae culture in batch and continuous mode operation.

FIG. 8 shows a graph of the productivity for a microalgae culture in batch and continuous mode operation.

FIG. 9 shows a graph of the nitrate concentration for a microalgae culture in batch and continuous mode operation.

FIG. 10 shows a graph of the dry weight for a microalgae culture in batch and continuous mode operation.

FIG. 11 shows a graph of the productivity for a microalgae culture in batch and continuous mode operation.

FIG. 12 shows a graph of the sodium nitrate and acetate uptake for a microalgae culture in batch and continuous mode operation.

FIG. 13 shows a graph of the dry weight for microalgae cultures inoculated with cells from the continuous mode operation.

FIG. 14 shows a graph of the dry weight for a microalgae culture in batch and continuous mode operation.

FIG. 15 shows a graph of the productivity for a microalgae culture in continuous mode operation.

FIG. 16 shows a graph of the acetic acid utilized for a microalgae culture in continuous mode operation.

FIG. 17 shows a graph of the acetic acid consumed and production for a microalgae culture in batch and continuous mode operation.

FIG. 18 shows a graph of the nitrogen concentration for a microalgae culture in batch and continuous mode operation.

FIG. 19 shows a graph of a culture dry weight for microalgae cultures in batch and continuous mode operation.

FIG. 20 shows a graph of culture productivity for microalgae cultures in batch and continuous mode operation.

FIG. 21 shows a graph of production and acetic acid consumption for microalgae cultures in continuous mode operation.

FIG. 22 shows a graph of production and nitrate consumption for microalgae cultures in continuous mode operation.

FIG. 23 shows a graph of a culture dry weight for microalgae cultures in semi-continuous and continuous mode operation.

FIG. 24 shows a graph of productivity for microalgae cultures in semi-continuous and continuous mode operation.

FIG. 25 shows a graph of production and acetic acid consumption for microalgae cultures in continuous mode operation.

FIG. 26 shows a graph of production and nitrate consumption for microalgae cultures in continuous mode operation.

FIG. 27 shows a graph of a culture dry weight for microalgae cultures in semi-continuous and continuous mode operation.

FIG. 28 shows a graph of productivity for microalgae cultures in semi-continuous and continuous mode operation.

FIG. 29 shows a graph of percent protein for microalgae cultures in semi-continuous and continuous mode operation.

FIG. 30 shows a graph of a culture dry weight for microalgae cultures in and continuous mode operation.

FIG. 31 shows a graph of productivity for microalgae cultures in continuous mode operation.

FIG. 32 shows a graph of a culture dry weight for microalgae cultures in semi-continuous and continuous mode operation.

FIG. 33 shows a graph of productivity for microalgae cultures in semi-continuous and continuous mode operation.

FIG. 34 shows a graph of a protein percent for microalgae cultures in semi-continuous and continuous mode operation.

FIG. 35 illustrates an exemplary embodiment of a method.

### DETAILED DESCRIPTION

Described in this specification are non-limiting embodiments for culturing microorganisms, such as microalgae and cyanobacteria, in a continuous auxostat system that operates in mixotrophic culture conditions. Benefits of the continuous mixotrophic method and system embodiments, and differences between continuous and batch or semi-continuous methods and systems are also described. Throughout the specification, the term “continuous” is used to describe activity that occurs multiple times within a short time period, such as multiple times within a minute, quarter hour, half hour, or hour.

Non-limiting examples of mixotrophic microalgae and cyanobacteria that may be used in the described continuous system and method embodiments comprise organisms of the genera: Agmenellum, Amphora, Anabaena, Anacystis, Apistonema, Arthrospira (Spirulina), Botryococcus, Brachiomonas, Chlamydomonas, Chlorella, Chloroccum, Cruciplacolithus, Cylindrotheca, Coenochloris, Cyanophora, Cyclotella, Dunaliella, Emiliania, Euglena, Extubocellulus, Fragilaria, Galdieria, Goniotrichium, Haematococcus, Halochlorella, Isochyrsis, Leptocylindrus, Micractinium, Melosira, Monodus, Nostoc, Nannochloris, annochloropsis, Navicula, Neospongiococcum, Nitzschia, Odontella, Ochromonas, Ochrosphaera, Pavlova, Picochlorum, Phaeodactylum, Pleurochyrsis, Porphyridium, Poteriochromonas, Prymnesium, Rhodomonas, Scenedesmus, Skeletonema, Spumella, Stauroneis, Stichococcus, Auxenochlorella, Cheatoceros, Neochloris, Ocromonas, Porphyridium, Synechococcus, Synechocystis, Tetraselmis, Thraustochytrids, Thalassiosira, and species thereof. Additionally, heterotrophic microalgae and cyanobacteria, such as but not limited to Schizochytrium/Aurantiochytrium, may be used in some embodiments of the described continuous system.

The organic carbon sources suitable for growing microalgae and cyanobacteria mixotrophically or heterotrophically, may comprise: acetate, acetic acid, ammonium linoleate, arabinose, arginine, aspartic acid, butyric acid, cellulose, citric acid, ethanol, fructose, fatty acids, galactose, glucose, glycerol, glycine, lactic acid, lactose, maleic acid, maltose, mannose, methanol, molasses, peptone, plant based hydrolyzate, proline, propionic acid, ribose, sacchrose, partial or complete hydrolysates of starch, sucrose, tartaric, TCA-cycle organic acids, thin stillage, urea, industrial waste solutions, yeast extract, and combinations thereof. The organic carbon source may comprise any single source, combination of sources, and dilutions of single sources or combinations of sources.

Non-limiting examples of suitable microalgae and cyanobacteria for mixotrophic growth using acetic acid or acetate as an organic carbon source may comprise organisms of the genera: Chlorella, Anacystis, Synechococcus, Synechocystis, Neospongiococcum, Chlorococcum, Phaeodactylum, Spirulina, Micractinium, Haematococcus, Nannochloropsis, Brachiomonas, and species thereof.

The term “pH auxostat” refers to the microbial cultivation technique that couples the addition of fresh medium (e.g., medium containing organic carbon such as acetic acid) to pH control. As the pH drifts from a given set point, fresh medium is added to bring the pH back to the set point. The rate of pH change is often associated to the consumption of a nutrient and meets the requirements as a growth-dependent parameter. The feed will replenish the residual nutrient, keeping the concentration relatively balanced with the buffering capacity of the medium. The pH set point may be modified to meet the requirements of the microorganisms present in the culture at the time. The microorganisms present may be driven by the location and season where the bioreactor is operated and how close the cultures are positioned to other contamination sources (e.g., other farms, agriculture, ocean, lake, river, waste water).

In continuous operation the fresh medium is typically more diluted than in semi-continuous operation (e.g. 20-0.1% acetic acid instead of 20-80% acetic acid). The cell density of the culture is controlled by the concentration of the nutrient that drives the pH drift (e.g., acetate, acetic acid).

The terms “microbiological culture”, “microbial culture”, or “microorganism culture” refer to a method or system for multiplying microorganisms, such as microalgae and cyanobacteria, through reproduction in a predetermined culture medium, including under controlled laboratory conditions. Microbiological cultures, microbial cultures, and microorganism cultures are used to multiply the organism, to determine the type of organism, or the abundance of the organism in the sample being tested. In liquid culture medium, the term microbiological, microbial, or microorganism culture generally refers to the entire liquid medium and the microorganisms in the liquid medium regardless of the vessel in which the culture resides. A liquid medium is often referred to as “media”, “culture medium”, or “culture media”. Nutrients in microorganism culture media may comprise nitrogen, phosphorus, micronutrients, trace metals, and vitamins. Many recipes for culture media can be found in the public domain, such as BG-11 media and f/2 media. The act of culturing is generally referred to as “culturing microorganisms” when emphasis is on plural microorganisms. The act of culturing is generally referred to as “culturing a microorganism” when importance is placed on a species or genus of microorganism. Microorganism culture is used synonymously with culture of microorganisms.

The term “axenic” describes a culture of an organism that is entirely free of all other “contaminating” organisms (e.g., unwanted organisms, organisms that are detrimental to the health of the microalgae or cyanobacteria culture). Throughout the specification, axenic refers to a culture that when inoculated in an agar plate with bacterial basal medium, does not form any colonies other than the microorganism of interest. Axenic describes cultures not contaminated by or associated with any other living organisms such as but not limited to bacteria, cyanobacteria, microalgae and/or fungi. Axenic is usually used in reference to pure cultures of microorganisms that are completely free of the presence of other different organisms. An axenic culture of microalgae or cyanobacteria is completely free from other different organisms.

Traditional culturing systems comprise either batch or semi-continuous operation. A batch culturing system comprises adding the amount of nutrients to a microorganism culture that is estimated that the microorganism culture will need over given period of time and then harvesting the culture at the end of the time period. The summary addition of nutrients at the beginning of the culturing process results in the culture media comprising more nutrients than the target microorganism can actively consume, and thus a residual concentration of nutrients is available for consumption by other contaminating organisms. The culture density in a batch operation will increase over the life of the culture until the entire culture is harvested. As the culture density increases, the availability of light for the microorganism cells decreases in the culture due to shading created by the increased number of cells in the same volume. A semi-continuous culturing system may comprise multiple discrete supplies of nutrients and multiple partial harvests of a microorganism culture over the life of the culture. The spaced addition of nutrients in a semi-continuous culture may still result in the residual nutrient concentration conditions found in a batch system, but culture density in a semi-continuous operation will only increase until the culture density is periodically reduced by partial harvests which may occur multiple times over the life of the microorganism culture.

Batch and semi-continuous systems have variable culture densities, growth phases, and stationary phases over periods of time between periodic partial harvests or from the start to a full harvest. Using a continuous system, the microorganisms can be sustained in a growth phase at a substantially constant density and/or productivity as a result of constant nutrient feeding and culture harvesting. Throughout the specification the term “substantially constant” means a variation of 20% or less (e.g., within plus or minus 20% of a value such as culture dry weight). Maintaining a substantially constant culture density facilitates control over the photosynthetic contribution to cell metabolism in a mixotrophic culture through light availability that is not achievable in batch or semi-continuous systems where availability of light decreases over time without additional lighting equipment or elaborate mixing regimes. A continuous auxostat system is also better configured to minimize residual nutrients accumulating in the culture from the nutrient supply to a culture of microorganisms in growth phase (i.e., on demand feeding). That nutrient supply is typically provided in excess under batch or semi-continuous system, which may facilitate the flourishing of populations of contaminating organisms or may be lost to waste upon harvesting and separating the microorganisms from the media. On demand feeding in a continuous auxostat system also minimizes the chances of the culture being limited in growth or productivity due to a nutrient deficiency that may occur in a suboptimal batch or semi-continuous operation.

By controlling the cell density in continuous operation, the mixotrophic growth rate can be optimized while controlling the relative contribution of either the photoautotrophic and heterotrophic metabolism. The higher growth achieved in continuous cultures maintained at an optimally balanced mixotrophic growth rate may be measured by the cell dry weights (i.e., biomass) or the production of target metabolites (e.g., pigments, lipids, proteins, carbohydrates, phytohormones), and may result in a higher average productivity than batch or semi-continuous cultures. The productivity advantage over time for a continuous system further increases when the time spent turning over and resetting systems that operate in a batch or semi-continuous mode is factored into the comparison. The number of times a continuous system needs to be taken down or reset is less than a batch or semi-continuous system. A continuous system described herein has also demonstrated an increased in culture longevity for closed continuous systems, with the increase longevity resulting at least 2 times, at least 3 times, at least 4 times, at least 5 times, or at least 6 times the number of viable days of culturing when compared to a batch or semi-continuous culture of the same species in mixotrophic conditions. Typical batch and semi-continuous cultures have a longevity of 7-14 days, while the continuous auxostat culture in the closed system of Example 9 demonstrates the capability to operate for at least 47 days, and may operate for at least 50 days, at least 55 days, at least 60 days, at least 70 days, at least 80 days, at least 90 days, or at least 100 days. Operation of a microorganism culture in continuous conditions may also increase the health of the cells and the amount of measureable, protein, lipids, polysaccharides, or phytohormones as compared to batch or semi-continuous conditions.

A continuous system may also provide efficiencies over a batch or semi-continuous system from capital and operating expenditure views, such as but not limited to: a reduction in labor for culturing vessel (i.e., bioreactor) operation, harvesting and media preparation; a reduction in the nutrient material used to produce a quantity of biomass; a reduction in the number of culturing vessels to produce a quantity of biomass; and the areal productivity for a culturing system. A continuous system may also reduce or eliminate the negative impact of conditions associated with high density cultures, such as but not limited to: the accumulation of foam which blocks light from reaching the microorganism culture and may provide a harbor for contaminating organisms; increased internal pressure of closed bioreactors which may lead to mechanical failure (e.g., break, tear, or rupture of bag bioreactor wall); shading of microorganisms by other microorganisms in the culture which leads to a reduction in light available to some cells for photosynthetic activity; the depletion of dissolved oxygen to levels that limits the metabolization of organic carbon by the cells and growth of the cells; and the accumulation of stress causing substances (e.g., malic acid) which lead to a decline in the culture health. In some embodiments, the increase in contaminating organisms (e.g., bacteria, fungi) in the microorganism culture of a continuous system may be linear over time, as opposed to the exponential increase experienced in batch and semi-continuous cultures, which provides different opportunities for treatment, mitigation, or prevention.

A continuous system may also provide means to naturally select for the fastest growing microorganism populations in a culture over time, which may result in an increase in productivity over time. A typical batch or semi-continuous culture does not have the selective pressure nor the longevity, but this selective pressure may be enabled in a continuous system due to the ability to maintain the same culture in growth phase for longer periods of time.

A continuous system may also provide the opportunity to efficiently incorporate recycled media. The spent media from a continuous system may be stabilized and preserved by using the acetic acid concentration that is typically used for continuous operation (e.g. 0.5-20%). Such acetic acid would inhibit microbial activity of the centrate for days or weeks during storage, allowing the centrate from previous harvest to be utilized in a subsequent culture when needed. The increased productivity in a continuous system may also carry over into a batch or semi-continuous system when the harvested culture is transferred from the continuous system to a batch or semi-continuous system.

Development of a continuous system for mixotrophic cultivation as described herein was initiated to find a way to control the amount of light available to cells in order to regulate the contribution of photosynthetic activity for mixotrophic growth. Previous experiments had shown that results achieved through heterotrophic growth may be exceeded by adding a photosynthetic contribution in mixotrophic conditions. The inventors identified the increase in culture density over time as a factor that limits the availability of light to the cells, and thus the contribution of photosynthesis to growth. Additional factors for developing the continuous system included the desire to increase the system efficiency for culturing microorganisms through the reduction of labor and downtime, as well as a desire to reduce costs associated with a culturing system on a volumetric or areal production basis.

While a continuous system can address these challenges, it was found by the inventors that not all continuous systems operate in the same manner or are equal in effectiveness for addressing the identified challenges. A turbiostat continuous system uses a variable flow rate, but requires feedback between the measured turbidity of the culture and the dilution rate to maintain a substantially constant culture density in the culturing vessel. A chemostat continuous system uses a continuous flow of fresh media continuously added, and the continuous removal of culture media containing residual nutrients, metabolic products, and microorganisms at the same rate to maintain a substantially constant culture volume. Continuous chemostat systems operate at predetermined dilution rates for operation and do not provide the capability for feeding on demand to reduce the likelihood of growth limitation from nutrient deficiency. Also, a continuous chemostat system could result in washing off the culture (i.e., reducing the culture density too far below the target) if the dilution rate approached the maximum growth rate of the organisms for the given conditions. The inventors developed a continuous auxostat system which utilizes feedback from a pH measurement of the culture to control the media flow rate and enable on demand feeding to maintain a constant culture density without a predetermined dilution or removal rate. Additionally, a continuous auxostat system will self-regulate the addition and harvest of culture media, and avoid washing off of the culture.

Development of the continuous auxostat system by the inventors comprised the calculation of assumptions for governing operation of the continuous system. Assumptions that may be calculated to determine the parameters for continuous operation comprise: the target productivity (g/L/day), target dry weight during steady continuous operation (g/L), target nitrogen yield (g of biomass/g of NaNO₃ or ammonium), target organic carbon yield (g of biomass/g of organic carbon consumed), evaporation rate (%/day), concentration of culture media nutrients, and concentration of organic carbon feed.

Generally, embodiments of a system for continuously culturing microorganism in mixotrophic conditions may comprise at least one culturing vessel, at least one pH auxostat system, at least one harvesting vessel, and harvesting means. In some embodiments, the at least one culturing vessel may comprise an open culturing vessel such as, but not limited to, a pond, a raceway pond, a trough, and other open bioreactors. In some embodiments, the at least one culturing vessel may comprise a closed culturing vessel such as, but not limited to, a bag, a tubular bioreactor, a tank, a flat panel bioreactor, or other closed bioreactors. In some embodiments, the at least one culture vessel may be configured to culture microorganisms in an aqueous culture medium. In some embodiments, the system may be disposed indoors. In some embodiments, the system may be disposed outdoors.

In some embodiments, the at least one pH auxostat system may be configured to provide fresh media with at least one of organic carbon and nutrients (e.g., nitrogen, phosphorus, trace metals, micronutrients) to the culture for at least one purpose selected from the group consisting of pH control, contamination control, and nutrition for sustaining growth. In other embodiments, the pH auxostat system may be configured to provide at least one nitrogen source, such as but not limited to ammonium, and nutrients to the culture for at least one purpose selected from the group consisting of pH control, contamination control, and nutrition for sustaining growth. The at least one pH auxostat system can measure the culture pH and provide new culture media comprising nutrients on demand to the culture of microorganisms, thereby facilitating the maintenance of the cells at the maximum growth rate with nutrients.

In some embodiments, the continuous system may be operated with microalgae and cyanobacteria capable of growth utilizing acetic acid or acetate, such as but not limited to, Chlorella, Anacystis, Synechococcus, Synechocystis, Neospongiococcum, Chlorococcum, Phaeodactylum, Spirulina, Micractinium, Haematococcus, Nannochloropsis, and Brachiomonas, and may supply acetate or acetic acid through the pH auxostat system. In some embodiments, the continuous system may be operated with microalgae and cyanobacteria capable of growth utilizing ammonium, such as but not limited to, Schizochytrium and Aurantiochytrium, and may supply ammonium through the pH auxostat system.

In some embodiments, the at least one harvesting vessel may be configured to receive at least a portion of the microorganism culture from the at least one culturing vessel. In some embodiments, the at least one harvesting vessel may be configured to receive the entire microorganism culture from the at least one culturing vessel. In some embodiments, the at least one harvesting vessel may be configured to provide agitation to the microorganism culture contained in the at least one harvesting vessel. In some embodiments, the at least one harvesting vessel may be configured to provide carbon dioxide to the microorganism culture contained in the at least one harvesting vessel. In some embodiments, the at least one harvesting vessel may be configured to provide organic carbon to the microorganism culture contained in the at least one harvesting vessel. In some embodiments, the at least one harvesting vessel may be configured to provide light to the microorganism culture contained in the at least one harvesting vessel. In some embodiments, the at least one harvesting vessel may be configured to provide organic carbon to the microorganism culture contained in the at least one harvesting vessel. In some embodiments, the at least one harvesting vessel may be configured to provide heat exchange (e.g., heating, cooling) to the microorganism culture contained in the at least one harvesting vessel. In some embodiments, the microorganisms may be sustained in a growth phase (i.e., continued cell growth and division) in the at least one harvesting vessel. In some embodiments, the microorganisms may be sustained in a stationary phase in the at least one harvesting vessel for the accumulation of a desired metabolite (e.g., pigment, lipid, protein, carbohydrate, phytohormones).

In some embodiments, the at least one harvest vessel may comprise an open container. In some embodiment, the at least one harvest vessel may comprise a closed container. In some embodiments, the at least one harvest vessel may comprise a bag, tank, tote, barrel, or other container. In some embodiments, the at least one harvest vessel may have a smaller volume than the at least one culturing vessel. In some embodiments, the at least one harvest vessel may have a larger volume than the at least one culturing vessel. In some embodiments, the at least one harvest vessel may be the same volume than the at least one culturing vessel. In some embodiments, the at least one harvest vessel may be sized based on the desired retention time for the harvested culture before transfer to another culturing vessel, culturing stage, or downstream processing.

In some embodiments, the harvesting means may comprise transferring the microorganism culture from the at least one culturing vessel to the at least one harvesting vessel by gravity, such as but not limited to, a transfer line with inlet and outlet at different elevations. In some embodiments, the harvesting means may comprise transferring the microorganism culture from the at least one culturing vessel to the at least one harvesting vessel by pumping action, such as but not limited to, peristaltic pumping. In some embodiments, the harvesting means may comprise check valves, seals, welds, or other known methods of mitigating the ingress of contaminating organisms (e.g., bacteria, fungi) into the harvesting means or at least one harvesting vessel.

In some embodiments, the microorganism culture may first be cultured without continuous harvesting or nutrient feeding (e.g., batch operation) in order to achieve a target dry weight, and then switched to run in the continuous operation mode (i.e., with continuous addition of new media containing nutrients and organic carbon, and harvesting of at least a portion of the culture) to maintain the target culture density and level of productivity. In some embodiments, the initial batch stage of operation and subsequent continuous stage of operation may be conducted in the same system comprising the at least one culturing vessel. In some embodiments, the initial batch stage of operation and subsequent continuous stage of operation may be conducted in the different systems comprising different culturing vessels. For embodiments where the stages are conducted in separate systems, the accumulated biomass from a batch operation may be harvested from a first culturing vessel and then inoculated at the target culture density into a second culturing vessel configured to operate as a continuous auxostat system. Whether in batch, semi-continuous, or continuous operation, the conditions for culturing microorganisms mixotrophically may comprise the infusion of gas containing oxygen, the supply of light, and the supply of an organic carbon source (e.g., acetate, acetic acid, glucose) to the culturing vessel. The means of supplying gas to an open or closed culturing vessel may comprise any means known in the art, such as but not limited to, air spargers, perforated tubing, gas injection nozzles, and bubble or microbubble generators. The means of supplying light may comprise any means known in the art, such as but not limited to, natural sunlight, light emitting diodes (LED), fluorescent bulbs, and incandescent bulbs). Heterotrophic cultivation in an embodiment of the continuous auxostat methods and system will not require a supply of light.

In one non-limiting embodiment, a continuous auxostat system comprising a closed culturing vessel may be used in seed stage cultivation (i.e., low volume cultures that will be used to inoculate subsequent larger volume cultures) for the production of microorganisms that will inoculate subsequent cultures. For example, a primary closed culturing vessel of a continuous auxostat system may continuously produce quantities of biomass to inoculate a group of batch or semi-continuous secondary culturing vessels with biomass of a consistent quality. Such closed culturing vessels may comprise, but are not limited to, bag, tubular, flat panel, and tank bioreactors. Non-limiting exemplary assumptions for operation of a continuous auxostat culturing system comprising a closed culturing vessel may comprise: a target cell culture density (i.e., dry weight) during steady dry weight of 15-30 g/L; a target acetic acid yield of 0.2-0.4 g of biomass/g of acetic acid; a target nitrate yield of 2-4 g of biomass/g of NaNO₃; an estimated evaporation rate of 5-20% per day; a concentration of nutrient media of 2-5 times the convention recipe concentration; and concentration of the acetic acid in the pH auxostat feed of 4-8%. A continuous auxostat system comprising closed bag bioreactors and a culture of Chlorella in acetic acid fed mixotrophic conditions has been shown to achieve productivities of at least 1 g/L/day, at least 2 g/L/day, at least 3 g/L/day, at least 4 g/L/day, and at least 5 g/L/day; an acetic acid yield of at least 0.10 g of biomass/g of acetic acid, at least 0.15 g of biomass/g of acetic acid, at least 0.20 g of biomass/g of acetic acid, at least 0.25 g of biomass/g of acetic acid, and at least 0.30 g of biomass/g of acetic acid; and a nitrate yield of at least 1.5 g of biomass/g of NaNO₃, at least 2.0 g of biomass/g of NaNO₃, at least 2.5 g of biomass/g of NaNO₃, at least 3.0 g of biomass/g of NaNO₃, and at least 3.5 g of biomass/g of NaNO₃. Productivities and yields of non-Chlorella organisms may be higher in embodiments of the continuous auxostat system.

In another non-limiting embodiment, a continuous auxostat system comprising an open culturing vessel may be used as a final stage of production. Such open culturing vessels may comprise, but are not limited to, raceway pond and trough bioreactors. For open culturing vessels the ability to control evaporation is not available in the same capacity as closed culturing vessels, and thus considerations for the impact of evaporation will be specific to the open culturing vessel used as well as the environmental conditions (e.g., temperature, humidity). Non-limiting exemplary assumptions for operation of a continuous auxostat culturing system comprising an open culturing vessel may comprise: a target cell culture density (i.e., dry weight) during steady dry weight of 2-8 g/L; a target acetic acid yield of 0.2-0.5 g of biomass/g of acetic acid; a target nitrate yield of 2-4 g of biomass/g of NaNO₃; a concentration of nutrient media of 0.5-3 times the convention recipe concentration; and concentration of the acetic acid in the pH auxostat feed of 0.5-4.0%. A continuous auxostat system comprising open raceway ponds and a culture of Chlorella in acetic acid fed mixotrophic conditions has been shown to achieve productivities of at least 0.5 g/L/day, at least 1.0 g/L/day, at least 1.5 g/L/day, at least 2.0 g/L/day, at least 2.5 g/L/day, and at least 3.0 g/L/day; an acetic acid yield of at least 0.20 g of biomass/g of acetic acid, at least 0.25 g of biomass/g of acetic acid, at least 0.30 g of biomass/g of acetic acid, at least 0.35 g of biomass/g of acetic acid, at least 0.40 g of biomass/g of acetic acid, at least 0.45 g of biomass/g of acetic acid, and at least 0.50 g of biomass/g of acetic acid; and a nitrate yield of at least 1.5 g of biomass/g of NaNO₃, at least 2.0 g of biomass/g of NaNO₃, at least 2.5 g of biomass/g of NaNO₃, at least 3.0 g of biomass/g of NaNO₃, and at least 3.5 g of biomass/g of NaNO₃.

In some embodiments, culturing microorganisms in a continuous auxostat system can provide a means for controlling the availability of light during mixotrophic cultivation. In one non-limiting embodiment, a method of controlling the availability of light during mixotrophic microalgae culturing may comprise: providing a culture of microorganisms in a culturing vessel at a predetermined culture density of a first dry weight per volume allowing a first availability of light within the culture of microorganisms and a daily biomass productivity for the microorganisms; supplying light to the culture of microorganisms; supplying fresh media to the microorganisms continuously; and removing a portion of the microorganism culture from the culturing vessel continuously, wherein the culture density of the microorganism culture in the culture vessel stays substantially constant or decreases to a dry weight per volume less than the first dry weight per volume over time with a corresponding availability of light greater than or equal to the first availability of light, and the daily biomass productivity remains substantially constant over time.

In some embodiments, the supply of light to the microorganism culture may be consistent, such as but not limited to, a consistent quantity, a consistent intensity, a repeated schedule of light and dark period, etc. In some embodiments, the supply of light to the microorganism culture may be variable, such as but not limited to, a changing intensity, flashing, etc. In some embodiments, the supply of fresh media may comprise at least one of organic carbon and nutrients. In some embodiments, the fresh media comprising organic carbon may be supplied by a pH auxostat system.

In some embodiments, culturing microorganisms in a continuous auxostat system can provide a means for selecting the highest productivity cells in a culture during the culturing process. In one non-limiting embodiment, a method for selecting for the highest productivity cells in a microorganism culture may comprise: providing a culture of microorganisms in a culturing vessel at a predetermined culture density of a first dry weight per volume and a daily biomass productivity; supplying fresh media to the culture of microorganisms continuously; and removing a portion of the microorganism culture from the culturing vessel continuously, wherein the culture density of the microorganism culture in the culturing vessel decreases to a least one second dry weight per volume less than the first dry weight per volume while the daily biomass productivity remains substantially constant over time to produce a microorganism culture of the highest biomass producing cells in the culturing vessel. In some embodiments, the method may further comprise supplying light to the microorganism culture. In some embodiments, the supply of fresh media may comprise at least one of organic carbon and nutrients. In some embodiments, the fresh media comprising organic carbon may be supplied by a pH auxostat system.

In some embodiments, a mixotrophic microorganism culture may be grown in a batch or semi-continuous operation stage and then switched to a continuous operation stage. In some embodiments, culturing a population of microorganisms in a batch or semi-continuous operation may comprise providing a discrete supply of organic carbon to raise the culture density from a first dry weight per volume to a second dry weight per volume. In some embodiments, productivity of a mixotrophic microorganism culture may be increased by switching from batch or semi-continuous operation to continuous operation. In some embodiments, the increase in productivity of a microorganism culture switched from batch or semi-continuous operation to continuous operation may be measured by the increased amount of at least one selected from the group consisting of biomass, protein, lipids, pigments, carbohydrates, and phytohormones over a given time period than a reference culture not continuously receiving fresh media and continuously removing a portion of the microalgae culture. In some embodiment, the increased amount of at least one selected from the group consisting of biomass, protein, lipids, pigments, carbohydrates, and phytohormones produced over a given time period may be at least 5%, at least 10%, at least 20%, at least 30%, at least 40%, at least 50%, at least 60%, at least 70%, at least 80%, at least 90%, or at least 100% more than the reference culture not continuously receiving fresh media and continuously removing a portion of the microalgae culture.

In some embodiments, longevity of a mixotrophic microorganism culture in a closed system may be increased by switching from batch or semi-continuous operation to continuous operation. In some embodiments, the increase in productivity of a microorganism culture switched from batch or semi-continuous operation to continuous operation may be measured by the increase in viable days of sustained microorganism cell growth, either numerically or as a multiple, than a reference culture not continuously receiving fresh media and continuously removing a portion of the microorganism culture. In some embodiments, the increase in sustained microorganism growth may be at least 1 day, at least 2 days, at least 3 days, at least 5 days, at least 10 days, at least 15 days, at least 20 days, at least 25 days, or at least 30 days longer than a reference culture not continuously receiving fresh media and continuously removing a portion of the microorganism culture. In some embodiments, the increase in sustained microorganism growth may be at least 2 times, at least 3 times, at least 4 times, at least 5 times, or at least 6 times longer than a reference culture not continuously receiving fresh media and continuously removing a portion of the microorganism culture.

In some embodiments, the seed stage of mixotrophic microorganism cultivation during a production process may occur in a continuous operation, with at least a portion of the culture from the continuous operation being transferred to inoculate a batch or semi-continuous operation in a production process. In one non-limiting embodiment, a method of continuously producing biomass for inoculating a batch or semi-continuous operation may comprise: mixotrophically culturing microorganisms at a substantially constant culture density in a closed first culturing vessel with the continuous supply of fresh media and continuous removal of a portion of the microorganism culture to a harvest vessel; and transferring at least some of the microorganism culture in the harvest vessel to a second culturing vessel which does not continuously supply fresh media and does not continuously remove a portion of the microorganism culture from the second culturing vessel wherein the culture density of the microorganism culture in the second culturing vessel is not substantially constant.

In some embodiments, the dilution rate of the microorganism culture in a continuous auxostat operation may not be predetermined or fixed. In some embodiments, the concentration of acetic acid and/or acetate in the fresh medium supplied in a continuous auxostat operation may contribute to controlling the culture density during continuous operation. In some embodiments, the concentration of acetic acid provided by the pH auxostat system may be in the range of 0.1% to 10%. In some embodiments, the concentration of acetic acid provided by the pH auxostat system may be in the range of 0.1% to 0.5%. In some embodiments, the concentration of acetic acid provided by the pH auxostat system may be in the range of 0.5% to 1.0%. In some embodiments, the concentration of acetic acid provided by the pH auxostat system may be in the range of 1.0% to 2.0%. In some embodiments, the concentration of acetic acid provided by the pH auxostat system may be in the range of 2.0% to 3.0%. In some embodiments, the concentration of acetic acid provided by the pH auxostat system may be in the range of 3.0% to 4.0%. In some embodiments, the concentration of acetic acid provided by the pH auxostat system may be in the range of 4.0% to 5.0%. In some embodiments, the concentration of acetic acid provided by the pH auxostat system may be in the range of 5.0% to 6.0%. In some embodiments, the concentration of acetic acid provided by the pH auxostat system may be in the range of 6.0% to 7.0%. In some embodiments, the concentration of acetic acid provided by the pH auxostat system may be in the range of 7.0% to 8.0%. In some embodiments, the concentration of acetic acid provided by the pH auxostat system may be in the range of 8.0% to 9.0%. In some embodiments, the concentration of acetic acid provided by the pH auxostat system may be in the range of 9.0% to 10.0%.

### EXAMPLES

Embodiments of the invention are exemplified and additional embodiments are disclosed in further detail in the following examples, which are not in any way intended to limit the scope of any aspect of the invention described herein. The strain of Chlorella used in the following examples provides an exemplary embodiment of the invention but is not intended to limit the invention to a particular strain of microalgae. Analysis of the DNA sequence of the exemplary strain of Chlorella in the NCBI 18s rDNA reference database at the Culture Collection of Algae at the University of Cologne (CCAC) showed substantial similarity (i.e., greater than 95%) with multiple known strains of Chlorella and Micractinium. Those of skill in the art will recognize that Chlorella and Micractinium appear closely related in many taxonomic classification trees for microalgae, and strains and species may be re-classified from time to time within the Chlorella and Micractinium genera. As would be understood in the art, the reclassification of various taxa is not unusual, and occurs as developments in science are made. Any disclosure in the specification regarding the classification of exemplary species or strains should be viewed in light of such developments. While the exemplary microalgae strain is referred to in the instant specification as Chlorella, it is recognized that microalgae strains in related taxonomic classifications with similar characteristics to the exemplary microalgae strain would reasonably be expected to produce similar results. Accordingly, Applicants submit that any mention of Chlorella in the specification and/or the claims would also be understood by a person of ordinary skill in the art to include Micractinium species genetically and/or morphologically similar to species classified within the genus Chlorella as of the filing date.

### Example 1

An experiment was conducted to determine if a closed bioreactor can operate in steady-state conditions using a continuous auxostat system for mixotrophic cultures of microalgae. Two closed bag bioreactors (i.e., a 90 L growth bag and 200 L harvest bag) were sterilized by autoclaving for 45 minutes at 121° C. The bioreactors were set side by side in a 4″ wide support frame and connected for fluid communication by silicon tubing axenically in a flow hood. Exhaust lines were connected to each bioreactor. The 90 L growth bag was axenically filled with a modified recipe of BG-11 culture media and one carboy containing a Chlorella seed culture. The culture of Chlorella was grown in mixotrophic conditions with 20% acetic acid/4% nitrate as the organic carbon and nitrogen sources in a batch mode until reaching a culture density of 4 g/L. The culture of Chlorella also received 200 μmol photon/m²/s light from each panel, and was mixed using an air sparger (40 Lpm) disposed in the base of the 90 L growth bag. A diluted formulation of BG-11 media with nutrients reduced down to match a 1.3% acetic acid concentration was prepared and stored in a 55 gallon drum of this media was prepared.

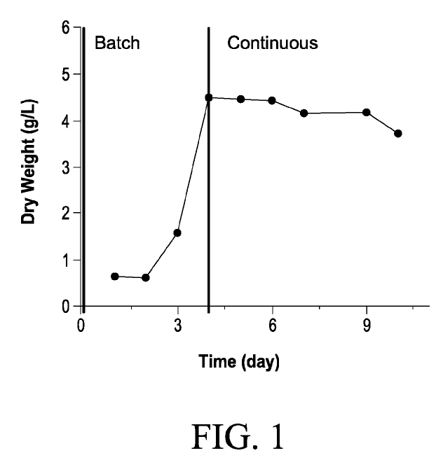

Experimental assumptions for the continuous culturing stage comprised: target cell culture density of 4 g/L, productivity of 2 g/L/day, continuous running volume of 90 L, and a daily harvest of 45 L. Upon the Chlorella culture reaching a 4 g/L density, the organic carbon feed supplied by the pH auxostat system was switched from a 20% acetic acid solution to the 1.3% acetic acid solution combined with nutrient media for continuous culturing mode. Tubing connecting the two bioreactors was configured in a fashion that allowed flow from the 90 L growth bag into the 200 L harvest bag by gravity. Each day, dry weight, nitrate and acetate samples were taken. Flow cytometry samples were taken as needed to quantify bacteria and algae populations. Additionally, the level (in inches) of the harvest bag and acid/media drum were measured and recorded. Any culture drained from the harvest bag was stored in 210 L drums with an additional 1.3% acetic acid. Results are shown in FIGS. 1-3.

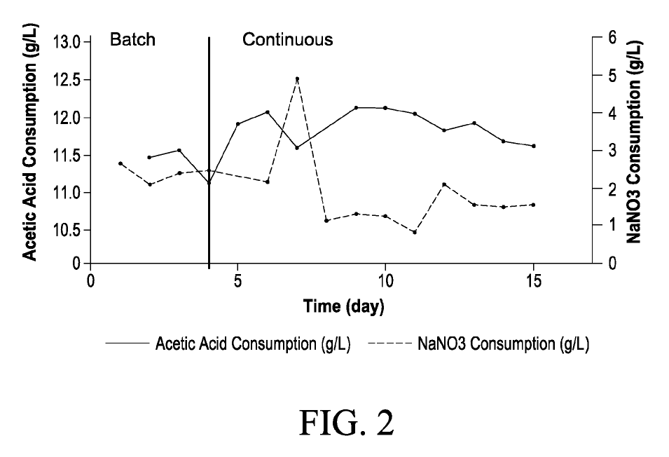

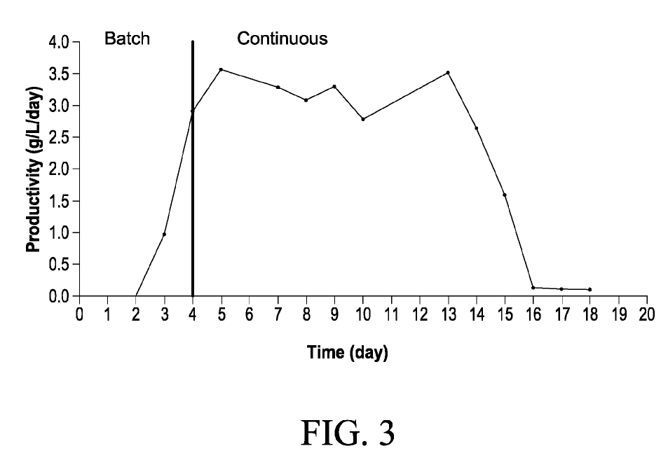

As shown in FIG. 1, the target culture density of 4 g/L was maintained in a continuous mode operation for four days until the bacteria population numbers increased and the culture pH was dropped in order to combat contamination. As shown in FIG. 2, the expected nutrient yields (acetate: 0.30, sodium nitrate: 2.5) corresponded to the actual nutrient yields acetate: 0.34 (avg.) and sodium nitrate: 2.80 (avg.). The average acetate and sodium nitrate yields were calculated in the continuous culturing mode during steady dry weight conditions. Nitrate uptake was lower than nitrate availability. As shown in FIG. 3, the productivity of −3.5 g/L/day was higher than the expected ˜2 g/L/day during the steady dry weight period between days 4 and 7. As the ratio of bacteria to microalgae increased, productivity fell. The impact of the bacterial contamination on the continuous culturing was shown as a decrease of productivity from 3.25 g/L/day to 0.91 g/L/day, a decrease in acetate yield from 0.35 to 0.20, and a decrease of nitrate yield from 1.05 to 0.71 after bacterial contamination occurred. The average daily harvest volume was 60-70 L, exceeding the assumption of 45 L/day. Thus, the results demonstrate continuous operation in a continuous auxostat system was achieved with mixotrophic cultures of microalgae.

### Example 2

An experiment was conducted to determine the feasibility of running a continuous auxostat system in an open bioreactor with a mixotrophic culture of microalgae. The open system consisted of an open raceway pond with paddlewheel mixing (25 Hz) disposed outdoors in a location capable of receiving light, a peristaltic pump to feed a liquid solution from the raceway pond to a 1,000 L tote (i.e., harvest tote) through silicon tubing, and a sump pump to feed a liquid solution from another 1,000 L tote to the raceway pond through silicon tubing. One tote was filled with 1,000 L of BG-11 culture media containing 0.9% (v/v) acetic acid as the organic carbon source and 0.13% (wt) of sodium nitrate. The open raceway pond was inoculated with a culture of Chlorella (1,118 L) and operated as a batch culture in mixotrophic conditions with a culture depth of 20 cm (i.e., BG-11 growth media, feed of 20% (v/v) acetic acid plus 2% (wt) of sodium nitrate). Once the culture reached a density of approximately 3 g/L, the system was transitioned to operate in continuous mode (i.e., draw the mixture of culture media and acetic acid from the tote to the raceway pond, and continuously transfer a portion of the culture in the raceway pond to a harvest tote). One end of silicon tubing was placed at the surface of the culture with the other end connected to a peristaltic pump operating at 12 rpm that fed the harvest tote to maintain the culture at a continuous volume level and density. The mixture of culture media and acid was fed to the culture in the raceway pond using a pH auxostat system with a set point of 7.5.

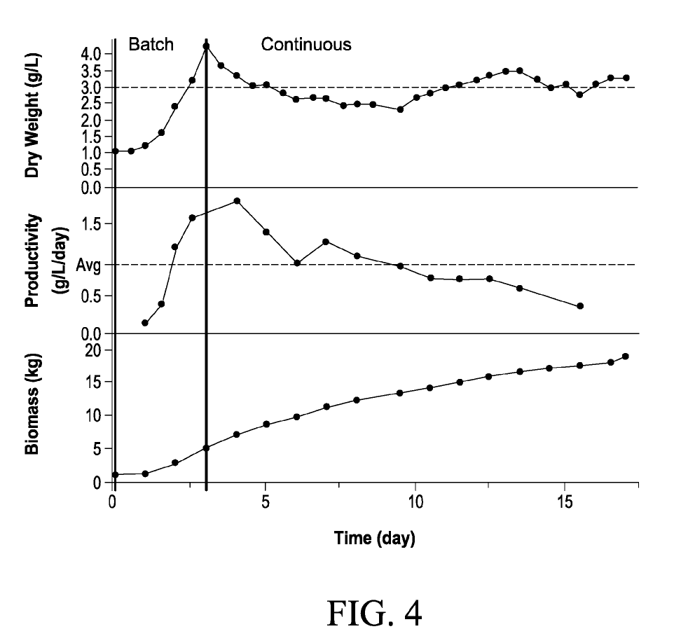

The culture temperature was maintained at 25° C. The supply of sunlight to the culture was variable due to cloud cover and weather. Dissolved oxygen level of the culture was adjusted daily through the supply of gases to the raceway pond to maintain a dissolved oxygen level of at least 2 mg/L. The level of the harvest tote was measured daily and used to calculate the productivity of the continuous system. Dry weight, nitrate, and acetate samples were taken twice per day. Petrifilm bacteria quantification, flow cytometry bacteria quantification, and phosphate samples were taken once per day. Harvest tote, raceway pond, and culture media tote measurements were taken every day in addition to microscope observations of the culture. Results are shown in FIGS. 4-6.

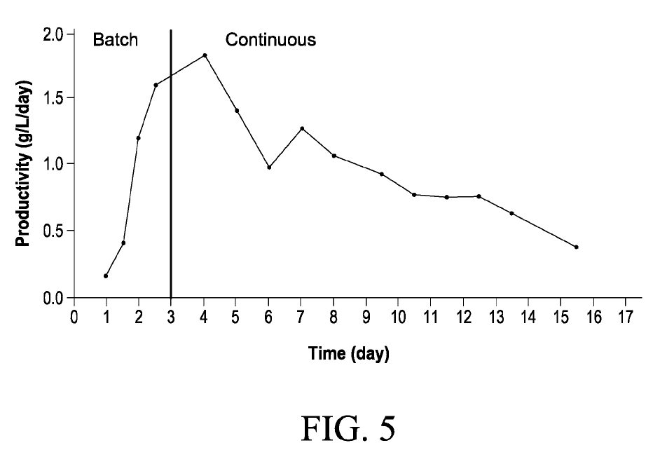

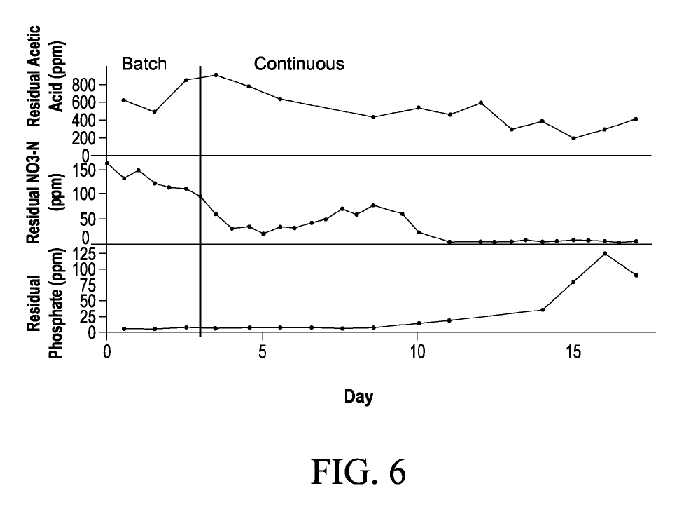

As shown in FIG. 4-5, the culture dry weight was maintained at a target range of 3 g/L for approximately 14 days during operation of the system in continuous mode. As shown in FIG. 6, excess nutrient concentrations in the culture were able to be kept to a minimum for a majority of the culture period. Nitrate levels were consumed and stabilized around 5 ppm. Phosphate levels exhibited a normal pattern of increasing over the duration of the culture period. The calculated sodium nitrate yield was 2.93 and the calculated sodium acetate yield was 0.32. Therefore, this experiment demonstrated the feasibility of operating an open mixotrophic culture of Chlorella in a continuous auxostat system.

### Example 3

An experiment was performed to determine the feasibility of operating a 200 L mixotrophic microalgae culture in a closed bioreactor utilizing a continuous auxostat operation. Two closed bag bioreactors with a 200 L culture volume capacity was outfitted with two stainless steel air spargers disposed within the bag bioreactor for mixing the culture of microalgae and supplying gases. One bioreactor (i.e., growth bag) was inoculated in a sterile manner with BG-11 culture media and Chlorella for mixotrophic cultivation. The culture was initially operated in batch mode (i.e., BG-11 and 40% [v/v] of acetic acid as the organic carbon source) until the culture reached a density of approximately 15 g/L. A 1,000 L tote with a mixture of culture media and 5.2% (v/v) acetic acid as the organic carbon source was used in continuous mode operation (i.e., after achieving 15 g/L, continuous transfer of the culture from the bioreactor to a harvest bag bioreactor through fittings and tubing, pH auxostat controlled feed of culture media and acetic acid to the bioreactor through fittings and tubing).

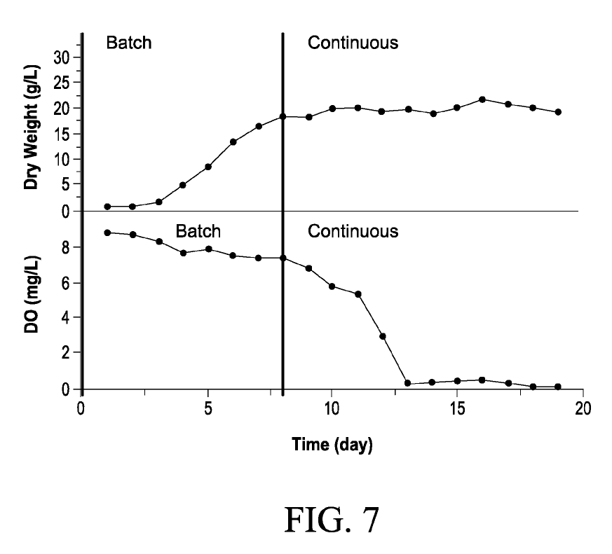

Dry weight, nitrate, and acetate samples were taken daily from the culturing bioreactor and harvest bioreactor. Microscope observations of both bioreactors were performed daily, flow cytometry bacteria quantification samples were taken periodically and daily measurements of the harvest bioreactor and culture media tote ware taking daily to calculate the culture productivity. The supply of air through the stainless steel spargers was adjusted as needed to prevent detrimental levels of foam. The results are shown in FIGS. 7-9.

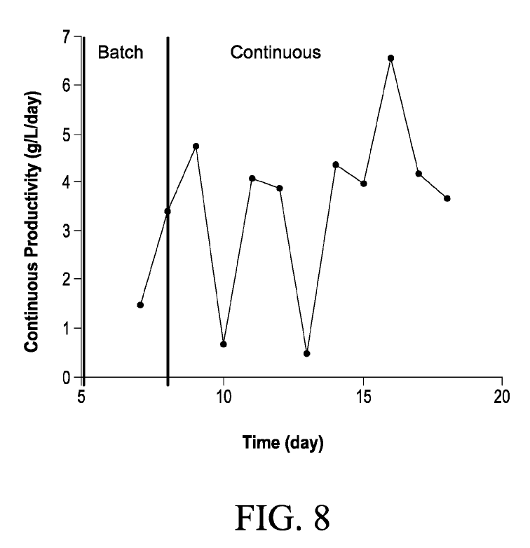

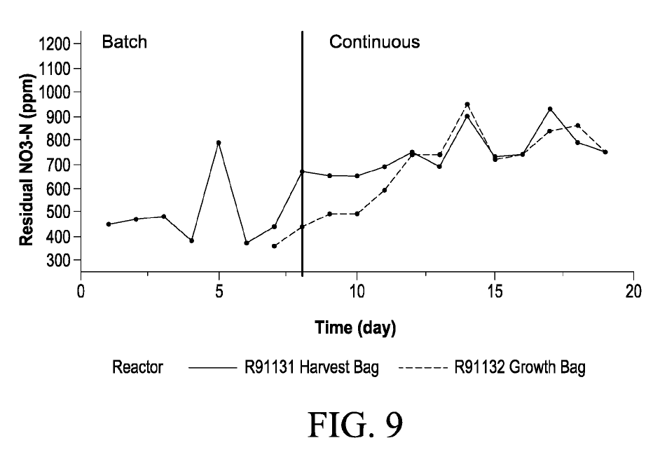

As shown in FIG. 7, a steady dry weight of approximately 20 g/L was attained during 12 days of continuous auxostat operation. The lower dry weight values may have been a result of an error in the projected evaporation of the cultures. As shown in FIG. 8, the level of foam peaked before days 10 and 13, making the calculated productivities for those days artificially low and the preceding days artificially high. The average productivity for the continuous culture period was 3.56 g/L day. As shown in FIG. 9, the nitrate levels in the harvest bioreactor closely mirrored those in the growth bioreactor, indicating that there is little growth occurring in the harvest bioreactor. Therefore, this experiment demonstrated the ability to achieve steady continuous operation of the mixotrophic microalgae culture in a closed bioreactor continuous auxostat system with a volume of at least 200 L.

### Example 4

An experiment was performed to determine if a closed bioreactor operating as a continuous auxostat can provide inoculum cultures of mixotrophic microalgae for a plurality of bioreactors. Two closed bag bioreactors with a 200 L culture volume capacity were autoclaved for reuse and outfitted with two stainless steel air spargers disposed within the bag bioreactor for mixing the culture of microalgae and supplying gases. One bioreactor (i.e., growth bag) was inoculated in a sterile manner with BG-11 culture media and Chlorella at a culture density of 0.5 g/L. The culture was initially operated in batch mode (i.e., BG-1 land 40% (v/v) of acetic acid as the organic carbon source and 4% (wt) sodium nitrate) until the culture reached a density of approximately 15 g/L. A 1,000 L tote with a mixture of culture media, 5.0% (v/v) acetic acid as the organic carbon source, and 0.23% (wt) sodium nitrate was used in continuous mode operation (i.e., after achieving 15 g/L, continuous transfer of the culture from the bioreactor to a harvest bag bioreactor through fittings and tubing, pH auxostat controlled feed of culture media and acetic acid to the bioreactor through fittings and tubing) at a target steady culture density of 20 g/L. Temperature was maintained at 28° C., and a pH set point of 7.5.

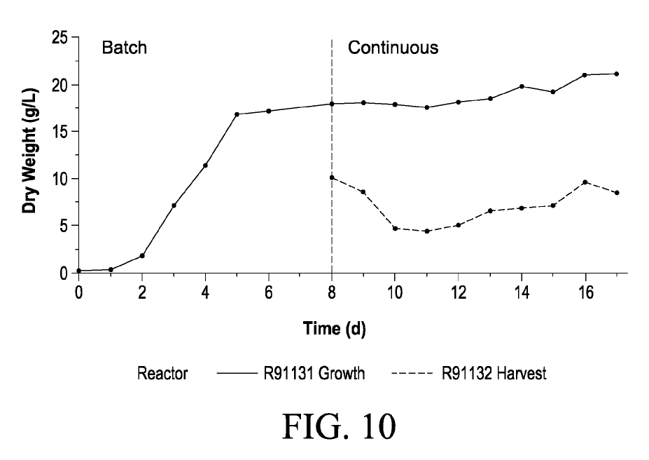

Dry weight, nitrate, and acetate samples were taken daily from the culturing bioreactor and harvest bioreactor. Microscope observations of both bioreactors were performed daily, flow cytometry samples were taken periodically and daily measurements of the harvest bioreactor and culture media tote ware taking daily to calculate the culture productivity. The supply of air through the stainless steel spargers was adjusted as needed to prevent detrimental levels of foam. The results are shown in FIGS. 10-13.

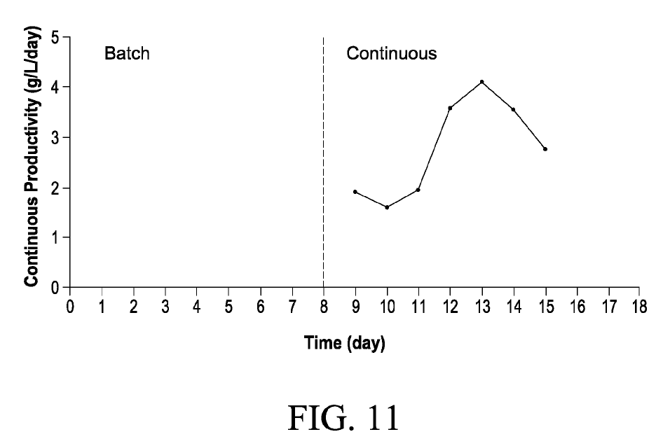

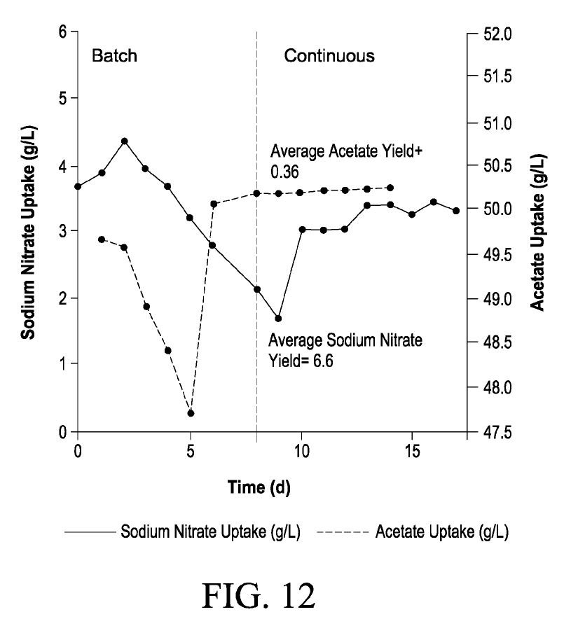

As shown in FIG. 10, the dry weight in the growth bioreactor was maintained at approximately 20 g/L for 8 days in continuous operation, meeting the calculated target in the experimental assumptions. As shown in FIG. 11, productivity in the continuous operation was variable (i.e., steady productivity was not reached due to foaming events), with a peak value of 4 g/L day. The ratio of bacteria to microalgae was low (i.e., below one) for the entire continuous culture period, suggesting that maintaining acceptable levels of contamination (e.g., axenic conditions) are feasible in a continuous mixotrophic operation of a closed bioreactor system. As shown in FIG. 12, uptake of acetate and sodium nitrate based on residual concentrations was relatively constant in continuous mode. The average acetate yield was 0.36, and the average sodium nitrate yield exceeded the value of about 2.5 seen in previous cultures with a value of 6.6.

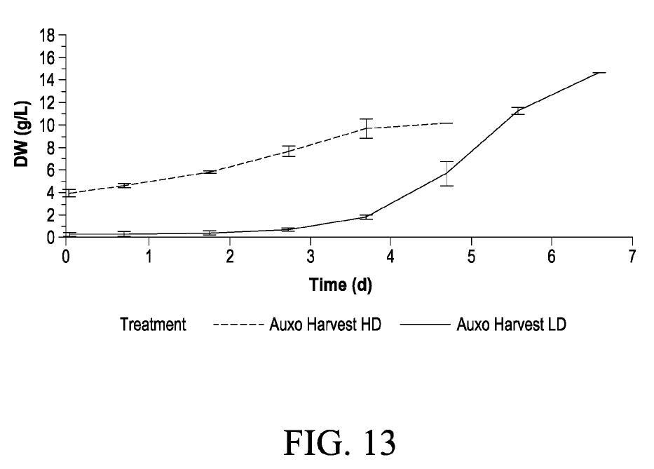

Additional bag bioreactors (i.e., stage 2) were inoculated with the culture resulting from the continuous mixotrophic operation. As shown in FIG. 13, both the bioreactors inoculated at a low density (LD) and a high density (HD) demonstrated growth using the inoculum from the continuous mixotrophic operation based on the dry weight (DW) measurements. The resulting cultures from the bioreactors at stage 2 also went on to inoculate more bag bioreactors (i.e., stage 3) and eventually an open raceway pond bioreactor (i.e., stage 4). This demonstrated that the microalgae cells produced in a continuous auxostat operation are capable of continued growth when transferred to subsequent bioreactor stages in mixotrophic conditions.

### Example 5

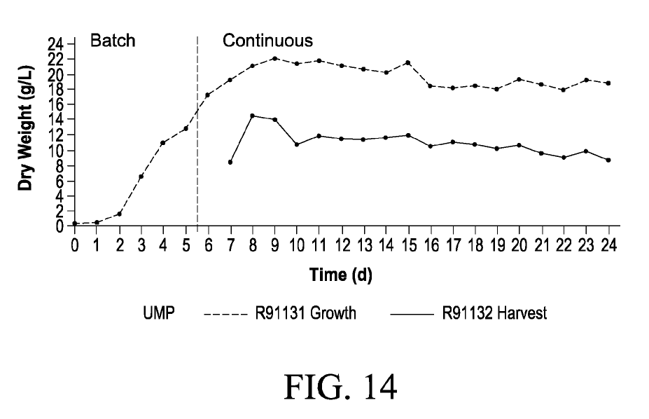

An experiment was conducted to repeat the results achieved in Example 4. The experiment was conducted as in Example 4 with the following changes in the protocol: an increased head space in the bag bioreactors, an increase in the culture volume to 220 L, and the acetic acid concentration in the culture media during continuous operation was increased to 6.1% (v/v). The mixotrophic culture was switched from batch to continuous auxostat operation at day 5.5. Results are shown in FIGS. 14-18

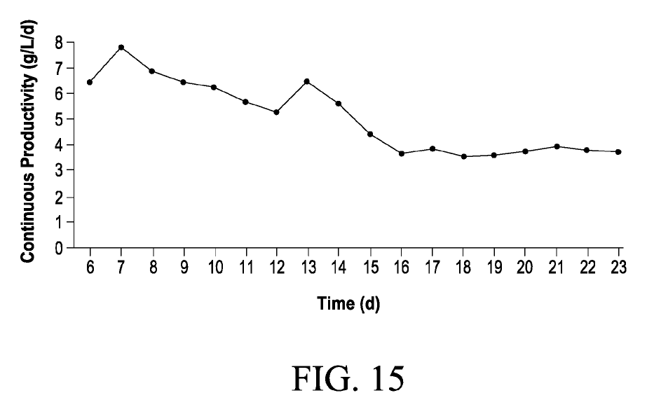

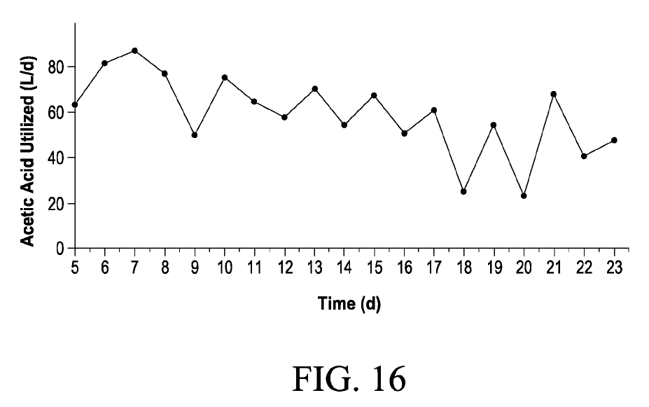

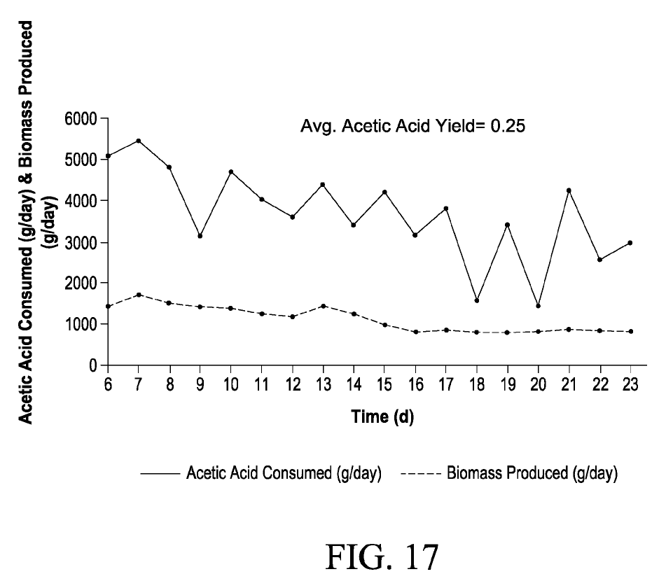

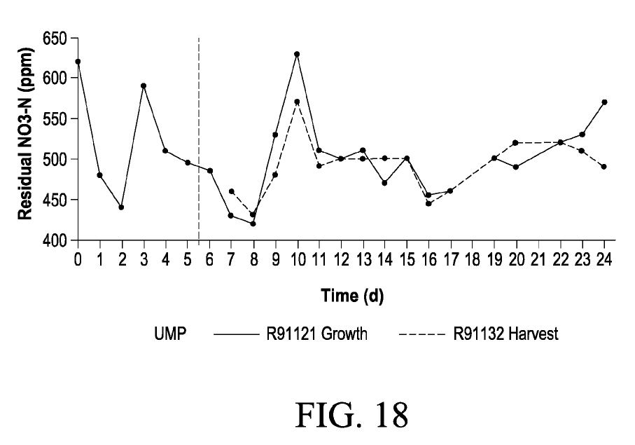

As shown in FIG. 14, the continuous operation ran successfully in axenic conditions for about 20 days with a dry weight above 18 g/L. A shown in the FIG. 15, the productivity peaked about day 7 (i.e., 1.5 days into the continuous mode operation) and produced an average productivity of 7.79 g/L day. As shown in FIG. 16, the acetic acid consumption during continuous operation was variable and demonstrated a slight reduction over time. As shown in FIG. 17, the average acetic acid yield for the continuous mode operation was 0.25. As shown in FIG. 18, the dotted line indicates the switch from batch to continuous operation, and shows the nitrogen concentration was also various during the culturing period. The resulting mixotrophic microalgae culture from the continuous auxostat operation was used to inoculate a subsequent bag bioreactor, and preformed comparably to reference inoculum produced from mixotrophic microalgae cultures in carboy bioreactors.

### Example 6

An experiment was conducted to compare the productivity of a batch culture and continuous auxostat culture of mixotrophic microalgae in an open outdoor bioreactor. The open system comprised an open raceway pond (culture depth of 20 cm) with submerged thrusters for mixing disposed outdoors in a location capable of receiving light; and for the continuous cultures also comprised a peristaltic pump to feed a liquid solution from the raceway pond to a tote (i.e., harvest tote) through silicon tubing, and a sump pump to feed a liquid solution from another tote to the raceway pond through silicon tubing. One tote was filled with BG-11 culture media containing 1.43% (v/v) acetic acid as the organic carbon source. The open raceway ponds were inoculated with a culture of Chlorella and operated as a batch culture in mixotrophic conditions (i.e., BG-11 growth media, batch feed of 40% [v/v] acetic acid plus 4% [wt] of sodium nitrate). For half of the cultures, the system was transitioned at day 3 to operate in continuous mode (i.e., draw the mixture of culture media and acetic acid from the tote to the raceway pond, and continuously transfer a portion of the culture in the raceway pond to a harvest tote) when the culture density reached approximately 5 g/L. The mixture of culture media and acid was fed to the continuous mode cultures using a pH auxostat system with a set point of 7.5. For all cultures pH was maintained at 6.5, and temperature was maintained at 25° C.

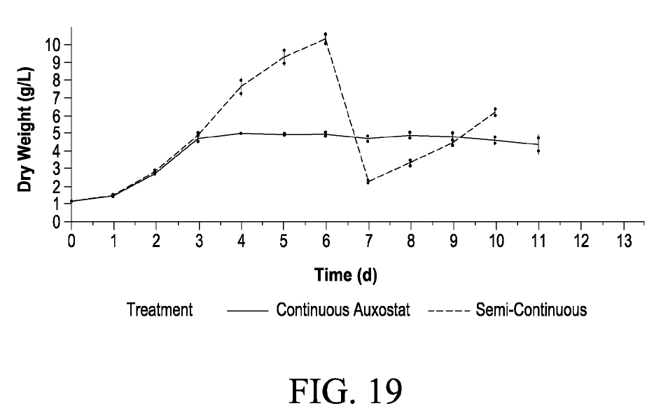

Samples were taken daily to assess culture dry weight, nitrate, acetic acid by High Performance Liquid Chromatography (HPLC), microscope observations, and flow cytometry quantification of bacteria. Parameters monitored at least twice a day included temperature, dissolved oxygen, and pH. Samples for protein analysis were collected initially, immediately before switching to continuous, 2 days after the switch, 4 days after the switch, and at the end of the experiment. Harvests of the batch mode culture occurred on days 6 and 10. Results are shown in FIGS. 19-22 for the 13 day culture period.

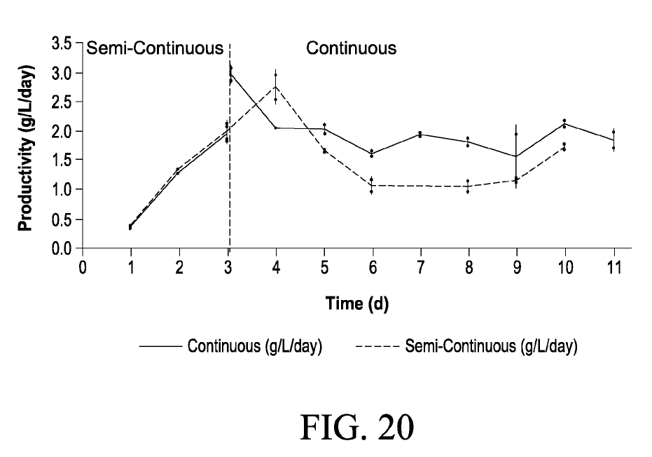

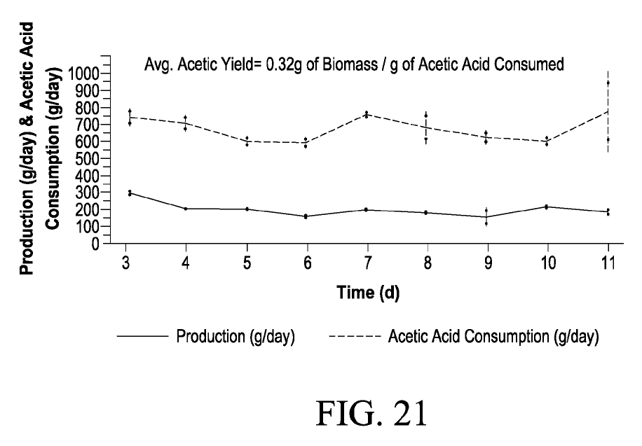

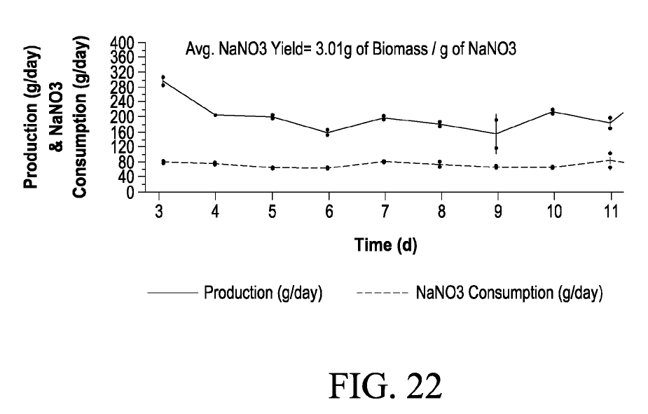

As shown in FIG. 19, average dry weight (DW) in continuous auxostat operation was 4.62 g/L which was substantially constant throughout the culturing period. As shown in FIG. 20, the average productivity of the cultures in continuous mode was 1.99 g/L day and 1.57 g/L day in batch mode, demonstrating 27% greater productivity for the culture operated in continuous mode. As shown in FIG. 21, the cultures operating in continuous mode had substantially constant production and acetic acid consumption, resulting in an average acetic acid yield of 0.32 g of biomass/g of acetic acid. Nitrates had to be batch fed to the cultures in continuous mode numerous times due to low nitrate levels and lack of a sufficient supply of nitrates from the BG-11 culture media. As shown in FIG. 22, the cultures operating in continuous mode had substantially constant production and nitrate consumption, resulting in an average nitrate yield of 3.01 g of biomass/g of NaNO₃.

Foam accumulation on the surface of the cultures was observed to be lower for the cultures operating in continuous mode, which allows for better access to light for the microalgae culture. Thus, the continuous auxostat operation demonstrated the ability to maintain a substantially constant culture density and the ability to outperform the productivity of a batch operation in an open outdoor mixotrophic system. The substantially constant production, acetic acid consumption, and nitrate consumption also demonstrates the reliability of the continuous auxostat operation in an open outdoor mixotrophic system.

### Example 7

An experiment was conducted to compare a semi-continuous and continuous auxostat mixotrophic microalgae operation in an open outdoor bioreactor. The outdoor bioreactor system, continuous mode operation, and type of microalgae (Chlorella) was the same as described in Example 6. The semi-continuous operation used 40% (v/v) acetic acid and 4% (wt.) sodium nitrate in a semi-continuous feed regime. All cultures began in semi-continuous operation and half of the cultures were switched to continuous mode operation on day five upon achieving a culture density of 7 g/L.

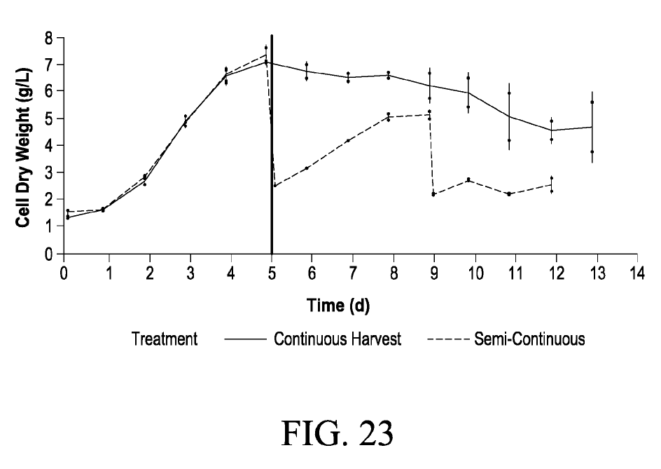

Samples were taken daily to assess culture dry weight, nitrate, acetic acid by High Performance Liquid Chromatography (HPLC), microscope observations, and flow cytometry quantification of bacteria. Parameters monitored at least twice a day included temperature, dissolved oxygen, and pH. Samples for protein analysis were collected initially, immediately before switching to continuous, 2 days after the switch, 4 days after the switch, and at the end of the experiment. Nitrate, acetic acid, and dry weight samples were collected immediately before every partial harvest of the semi-continuous cultures. Harvests of the batch mode culture occurred on days 5 and 9 for the semi-continuous cultures. Results are shown in FIGS. 23-26 for the 13 day culture period.

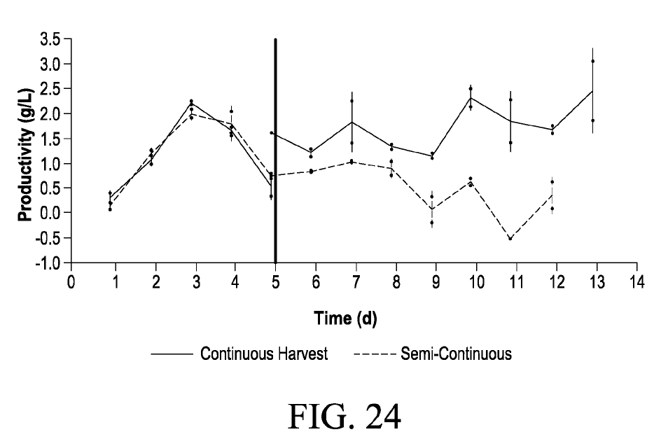

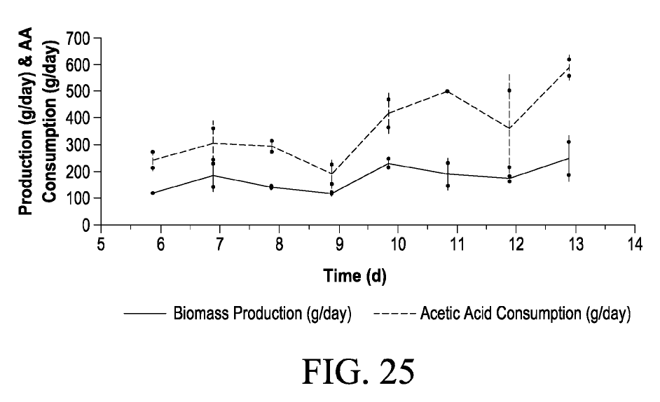

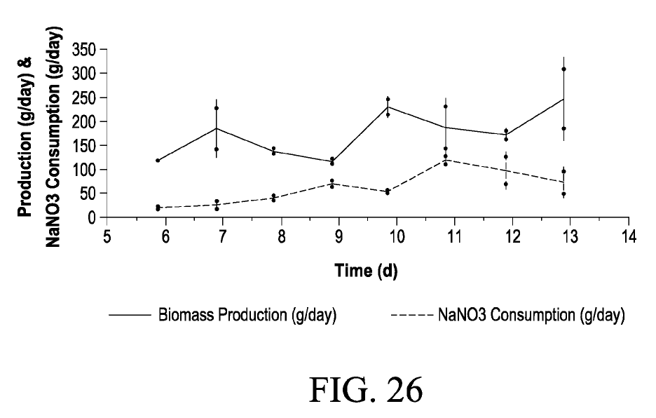

As shown in FIG. 23, average cell dry weight (CDW) for the continuous auxostat cultures was 5.77 g/L (+/−0.64 g/L). A shown in FIG. 24, the average productivity of the cultures in continuous mode was 1.4 g/L day (+/−0.2 g/L day) and 0.87 g/L day (+/−0.04 g/L day) in semi-continuous mode, demonstrating a 38% greater productivity for the cultures operated in continuous mode. As shown in FIG. 25, the average acetic acid yield for the cultures operating in continuous mode was 0.48 g of biomass/g of acetic acid consumed (+/−0.05 g/g). As shown in FIG. 26, the average acetic acid yield for the cultures operating in continuous mode was 2.91 g of biomass/g of sodium nitrate consumed (+/−0.04 g/g). A comparison of the semi-continuous and continuous mode operations for days 5-13 is shown in Table 1.

TABLE 1

| | Semi-Continuous | Continuous |
|---|---|---|
| Average Productivity | 1.47 ± 0.07 | 1.78 ± 0.16 |
| (g/L/day) | | |
| Total biomass harvested (g) | 1032 ± 48.08 | 1389 ± 123.5 |
| Total volume harvested (L) | 216.6 ± 0.35 | 251.43 ± 8.82 |
| Water usage (L/kg) | 209.60 ± 10.11 | 181.95 ± 22.52 |
| Average acetic acid yield (g | 0.35 ± 0.09 | 0.48 ± 0.05 |
| of biomass/g of AA | | |
| consumed) | | |
| Total acetic acid wasted in | 0.21 ± 0.7 | 0.17 ± 0.4 |
| supernatant (g AA/g DW) | | |
| Average acetic acid at | 0.99 ± 0.29 | 0.97 ± 0.33 |
| time of harvest (g/L) | | |
| Total acetic acid input (g) | 3288.95 ± 738.75 | 3125.33 ± 54.64 |
| Average NaNO₃ yield | 2.85 ± 0.67 | 2.31 ± 0.18 |
| (g of biomass/g of | | |
| NaNO₃ consumed) | | |
| Total NaNO₃ wasted in | 0.19 ± 0.4 | 0.09 ± 0.03 |
| supernatant (g of | | |
| NaNO₃/g DW) | | |
| Average NaNO₃ at time | 0.91 ± 0.25 | 0.49 ± 0.09 |
| of harvest (g/L) | | |
| Total NaNO₃ input (g) | 370.63 ± 70.36 | 600.92 ± 7.88 |

Thus, the continuous auxostat operation demonstrated better productivity than the semi-continuous operation for the open outdoor mixotrophic system. The continuous mode operation also required more nitrate, but had a better acetic acid yield and smaller water usage than the semi-continuous mode.

### Example 8

An experiment was conducted to compare a semi-continuous and continuous auxostat mixotrophic microalgae operation in an open outdoor bioreactor. The outdoor bioreactor system, protocols, and type of microalgae (Chlorella) was the same as described in Example 7. All cultures began in semi-continuous operation and half of the cultures were switched to continuous mode operation on day three upon achieving a culture density of 5 g/L.

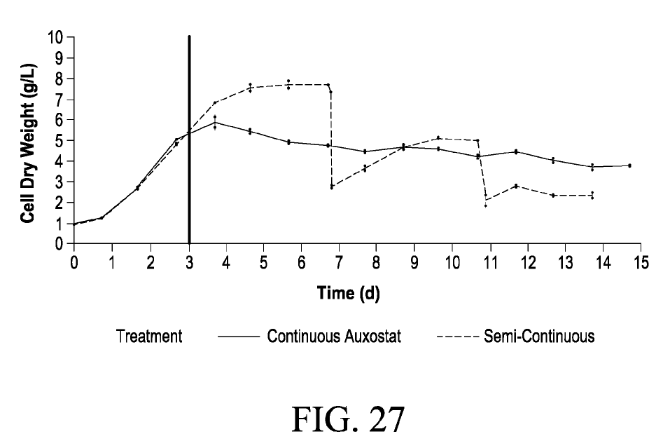

Samples were taken daily to assess culture dry weight, nitrate, microscope observations, and flow cytometry quantification of bacteria. Parameters monitored at least twice a day included temperature, dissolved oxygen, and pH. Samples for protein analysis were collected initially, immediately before switching to continuous, 2 days after the switch, 4 days after the switch, and at the end of the experiment. Nitrate, acetic acid, and dry weight samples were collected immediately before every partial harvest of the semi-continuous cultures. Harvests of the batch mode culture occurred on days 6 and 10 for the semi-continuous cultures. Results are shown in FIGS. 27-29 for the 15 day culture period.

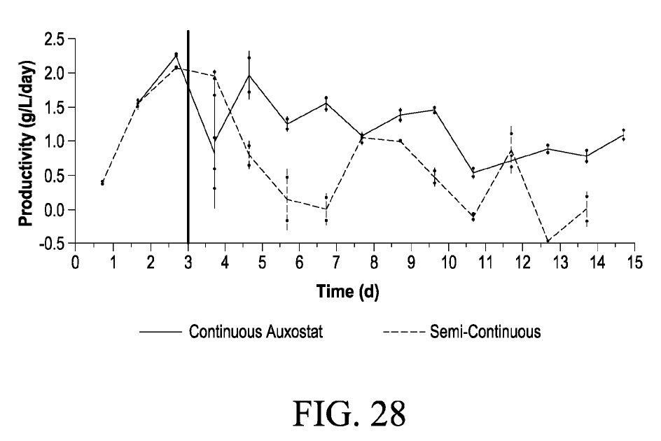

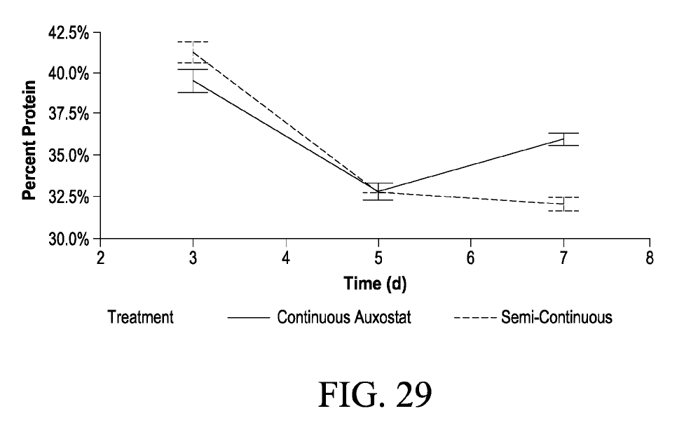

As shown in FIG. 27, average cell dry weight (CDW) for the continuous auxostat cultures was 4.56 g/L (+/−0.04 g/L). A shown in FIG. 28, the average productivity of the cultures in continuous mode was 1.19 g/L day (+/−0.02 g/L day) and 0.72 g/L day (+/−0.03 g/L day) in semi-continuous mode, demonstrating 39% greater productivity for the cultures operated in continuous mode. The average cell dry weight and productivity for the continuous mode operation also showed less variability than the cultures in Example 7. As shown in FIG. 29, the continuous mode cultures showed at increase in protein content over the semi-continuous mode cultures at day 7. A comparison of the semi-continuous and continuous mode operations for days 3-15 is shown in Table 2.

TABLE 2

| | Semi-Continuous | Continuous |
|---|---|---|
| Avg productivity (g/L/day) | 0.72 ± 0.03 | 1.19 ± 0.02 |
| Avg DW (g/L) | — | 4.56 ± 0.04 |
| Total biomass harvested (g) | 980 ± 50 | 1750 ± 30 |
| Total volume harvested (L) | 221 ± 6 | 398 ± 4 |
| Water footprint (L/kg) | 225 ± 5 | 228 ± 2 |
| Longevity (d) | 13.7 | 14.7 |
| Days after switch | 10.7 | 11.7 |
| DW at time of switch (g/L) | 4.82 ± 0.06 | 5.03 ± 0.02 |
| Avg acetic acid yield (g of biomass/g | 0.21 ± 0.05 | 0.22 ± 0.07 |
| of AA consumed) | | |
| Total acetic acid wasted in | 0.23 ± 0.01 | 0.17 ± 0.02 |
| supernatant (g AA/g DW) | | |
| Avg acetic acid at time of harvest | 1.03 ± 0.0 | 0.74 ± 0.09 |
| (g/L) | | |
| Total acetic acid input (g) | 5000 ± 900 | 9000 ± 2000 |
| Avg NaNO3 yield (g of biomass/g of | 0.4 ± 0.5 | 1.5 ± 0.4 |
| NaNO3 consumed) | | |
| Total NaNO3 wasted in supernatant | 0.46 ± 0.09 | 0.101 ± 0.002 |
| (g of NaNO3/g DW) | | |
| Avg NaNO3 at time of harvest (g/L) | 2.0 ± 0.5 | 0.44 ± 0.01 |
| Total NaNO3 input (g) | 1200 ± 200 | 1200 ± 300 |

Thus, the continuous auxostat mixotrophic operation demonstrated better productivity and produced more total biomass than the semi-continuous operation for the open outdoor mixotrophic system.

### Example 9

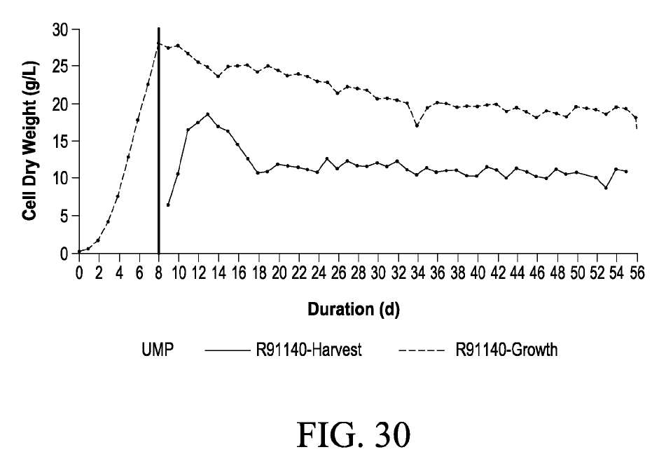

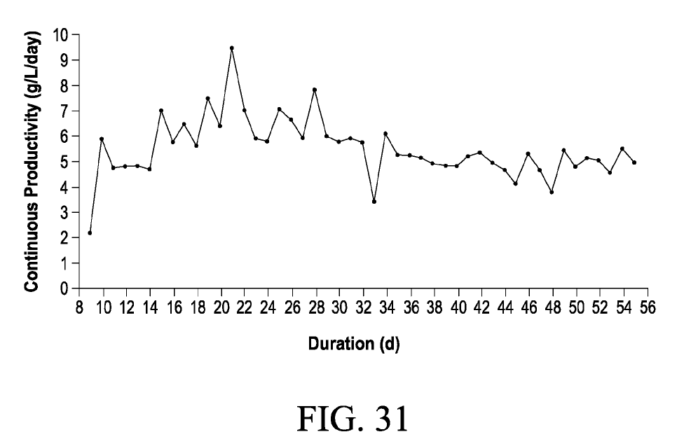

An experiment was conducted to determine if the continuous auxostat mixotrophic operation in a closed system can run for more than 30 days. The system was operated as in Example 5 with the following changes in the protocol: a decrease in the culture volume of the bioreactors to 90 L. The culture started in batch phase receiving 40% (v/v) acetic acid and 4% (wt.) NaNO₃, with phosphates doses when the cultures reached densities of 5 and 15 g/L. The cultures was transitioned to continuous operation when the culture density reached 27 g/L on day 8, comprising the continuous feed of media with 6.1% (v/v) acetic acid. Results are shown in FIGS. 30-31.

Samples were taken from the growth and harvest bags every day for dry weight, nitrate, acetic acid, and flow cytometry quantification of bacteria measurements. Microscope observations were also performed daily to track contamination. Daily measurements were also taken from the harvest bag and nutrient media tote to calculate the volume in and out for continuous productivity. The air flow rate was adjusted as needed to prevent foaming.

As shown in FIG. 30, the average dry weight for the continuous auxostat culture in the growth bioreactor was 21.55 g/L over the course of 47 days in continuous operation, with the culture in the harvest bioreactor maintaining a lower culture density. Typically, the longevity of batch or semi-continuous cultures is 7 to 14 days. As shown in FIG. 31, the productivity was relatively steady during continuous operation with an average of 5.05 g/L day. When viewing FIGS. 30 and 31 together, the cultures demonstrated the ability to maintain productivity as the culture density slowly decreased over time indicating that the continuous mode operation provides the ability to naturally select for the highest producing (i.e., fasting growing) cells in a culture (i.e., the end culture had a smaller number of cells producing the same biomass that a larger number of cells were producing in the beginning of the culture). The cell dry weight in the harvest bioreactor was observed to generally decrease over time from 14.5 g/L (day 24) to 11.6 (day 37), but the protein content increased from 19.7% (day 24) to 21.7% (day 37). Additionally, the continuous culture had an average acetic acid yield of 0.35 g of biomass/g of acetic acid consumed and an average NaNO₃ yield of 3.8 g of biomass/g of NaNO₃ consumed while demonstrating the ability to operate continuously in mixotrophic well beyond 30 days.

### Example 10

An experiment was conducted to compare a semi-continuous and continuous auxostat mixotrophic microalgae operation in an open outdoor bioreactor utilizing recycled media. The outdoor bioreactor system, protocols, and type of microalgae (Chlorella) was the same as described in Example 7. 100% filtered ozone treated separated culture media from a preceding mixotrophic culture was supplied to the semi-continuous culture after partial harvests. After the first partial harvest, filtered separated culture media from a preceding mixotrophic culture was treated with acetic acid and applied through the continuous auxostat feed to the continuous cultures. In the time between the continuous switch and the first partial harvest, conventional continuous feed media was used to maintain pH on the continuous cultures through the pH auxostat. All cultures began in semi-continuous operation and half of the cultures were switched to continuous mode operation on day four upon achieving a culture density of 5 g/L.

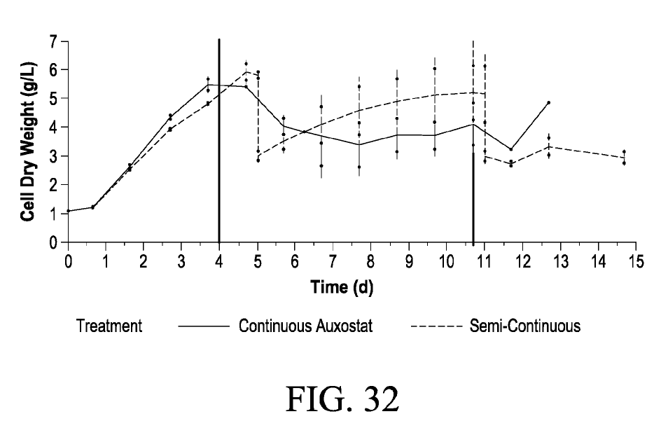

Samples were taken daily to assess culture dry weight, nitrate, microscope observations, and flow cytometry quantification of bacteria. Parameters monitored at least twice a day included temperature, dissolved oxygen, and pH. Samples for protein analysis were collected initially, immediately before switching to continuous, 2 days after the switch, 4 days after the switch, and at the end of the experiment. Nitrate, acetic acid, and dry weight samples were collected immediately before every partial harvest of the semi-continuous cultures. Harvests of the semi-continuous mode culture occurred on days 5 and 11 for the semi-continuous cultures. Results are shown in FIGS. 32-34 for the 15 day culture period.

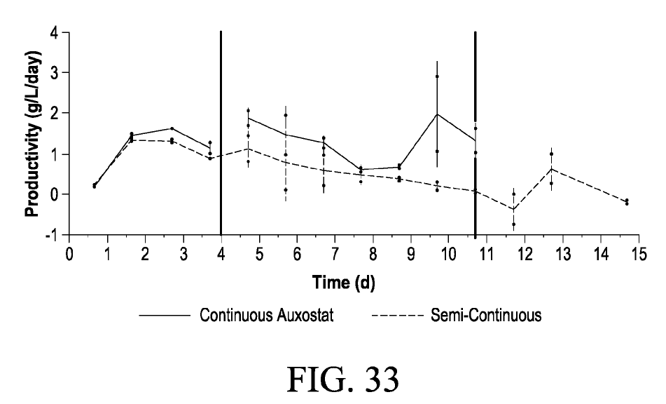

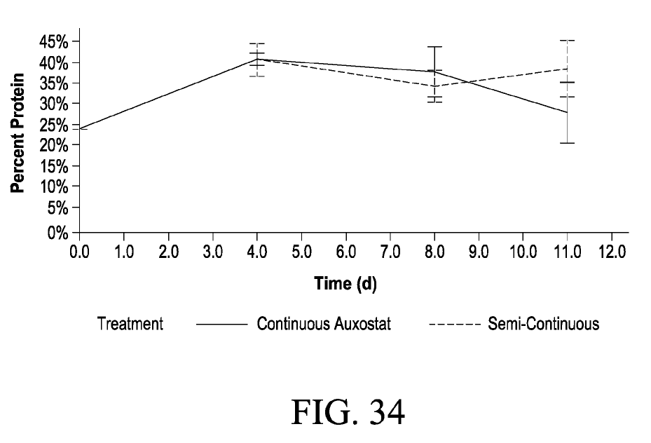

As shown in FIG. 32, the average cell dry weight (CDW) for the continuous auxostat culture was 4.9 g/L (+/−0.5 g/L). As shown in FIG. 33, the average productivity of the cultures in continuous mode was 1.14 g/L day (+/−0.06 g/L day) and 0.75 g/L day (+/−0.2 g/L day) in semi-continuous mode, demonstrating 39% greater productivity for the cultures operated in continuous mode. As shown in FIG. 34, the protein content of the continuous and semi-continuous cultures was comparable until day 11. A comparison of the semi-continuous and continuous mode operations for days 3-15 is shown in Table 3.

TABLE 3

| | Semi-Continuous | Continuous |
|---|---|---|
| Avg productivity (g/L/day) | 0.75 ± 0.2 | 1.14 ± 0.06 |
| Avg DW (g/L) | — | 4.9 ± 0.5 |
| Total biomass harvested (g) | 800 ± 200 | 1220 ± 60 |
| Total volume harvested (L) | 148 ± 5 | 310 ± 50 |
| Water footprint (L/kg) | 190 ± 30 | 250 ± 60 |
| Longevity (d) | 10.7 | 10.7 |
| Days after switch | 6.7 | 6.7 |
| DW at time of switch (g/L) | 4.81 ± 0.04 | 5.5 ± 0.3 |
| Avg acetic acid yield (g of biomass/g | 0.12 ± 0.01 | 0.20 ± 0.02 |
| of AA consumed) | | |
| Total acetic acid wasted in supernatant | 0.31 ± 0.08 | 0.35 ± 0.06 |
| (g AA/g DW) | | |
| Avg acetic acid at time of harvest (g/L) | 1.6 ± 0.1 | 1.4 ± 0.1 |
| Total acetic acid input (g) | 7000 ± 1000 | 6500 ± 300 |
| Avg NaNO3 yield (g of biomass/g of | 0.47 ± 0.03 | 1.05 ± 0.08 |
| NaNO3 consumed) | | |
| Total NaNO3 wasted in supernatant | 0.54 ± 0.06 | 0.41 ± 0.09 |
| (g of NaNO3/g DW) | | |
| Avg NaNO3 at time of harvest (g/L) | 2.9 ± 0.2 | 1.63 ± 0.01 |
| Total NaNO3 input (g) | 1700 ± 500 | 1170 ± 40 |

Based on these results, the continuous auxostat mode mixotrophic operation demonstrated better productivity, produced more total biomass, had a higher acetic acid yield, and had a higher nitrate yield than the semi-continuous operation for the open outdoor mixotrophic system.

### Example 11

An experiment was conducted to compare a semi-continuous and continuous auxostat mixotrophic microalgae operation in an open outdoor bioreactor utilizing recycled media. The outdoor bioreactor system, protocols, and type of microalgae (Chlorella) was the same as described in Example 7. Different treatments were applied to the centrate resulting from membrane filter separation. The continuous media was treated with 14 g/L acetic acid. Previous experiment carried out in well mixed (100 rpm) Erlenemeyer flasks showed that the centrate could be stabilized for long periods (>21 days) through the addition of at least 5 g/L acetic acid (see Table 4). The media used for semi-continuous after each partial harvest was treated with 100% filtered ozone and stabilized by refrigeration at 4° C. After the first harvest, continuous treatment started in two of the four reactors. In the time between the continuous switch and the first partial harvest, non-recycled continuous feed media was used to maintain pH on the continuous cultures through the pH auxostat. All cultures began in semi-continuous operation and half of the cultures were switched to continuous mode operation on day four upon achieving a culture density of 5 g/L. No bacterial growth was observed for the treatments of 5 g/L acetic acid and above for up to 21 days.

TABLE 4

| | 7 days | 14 days | 21 days |
|---|---|---|---|
| Acetic acid 0 g/L S | 0.00 | 0.542 | 0.79 |
| Acetic acid 5 g/L S | 0.00 | 0.00 | 0.00 |
| Acetic acid 10 g/L S | 0.00 | 0.00 | 0.00 |
| Acetic acid 15 g/L S | 0.00 | 0.00 | 0.00 |

### Example 12

An experiment was conducted to balance the mixotrophic metabolisms in the acidophilic microalgae Galdieria sulphuraria. The relative contribution of photoautotrophic and heterotrophic metabolism is a key parameter in the technoeconomic and life cycle assessment of any mixotrophic process. Therefore, this experiment aims to develop a method to grow mixotrophic microalgae while controlling the relative contribution of each metabolism. The mixotrophic balance could be affected by several factors such as shelf shading, photoperiod, weather, light path, substrate concentration and its catabolic repression. Accordingly, we design a continuous system able to self-regulate in response to changes in photosynthetic activity and modulate its growth rate to match our target substrate yields.

Galdieria sulphuraria was inoculated in a continuous ammonium/pH-auxostat system. The ammonia concentration in the feedstock (0.8 g/L) was used to control the steady state density (5 g/L), as estimated from nitrogen source yields of this species (6.2). The system was supplemented with other inorganic nutrients (g/L); ammonium sulfate (5.8), magnesium sulfate heptahydrate (0.3), monosodium phosphate (0.3), calcium chloride (0.02), sodium chloride (0.02), Fe-EDTA solution (2 ml/L) and trace metal solution (2 ml/L). The continuous media was also supplemented with glucose as organic carbon source. The glucose to ammonia ratio of the feedstock was used to target a specific substrate yield. In this experiment, the media was supplemented with 8 g/L glucose with the aim of controlling o maintain photosynthetic growth contribution at 25%.

The continuous culture grew at a constant 1 g/L d and maintained substrate yields at 1. Changes in illumination rate between 200 and 400 micromol photon/m2 sec did slightly decrease productivities, but as expected did not impact the steady state density or the substrate yields. This yield (1 g biomass/g os substrate) represents a 30% improvement from the heterotrophic yields (0.7) typically observed under similar conditions, suggesting that the system maintains 30% of photosynthetic growth as initially targeted by the process. The off-gas analysis confirmed the balance between heterotrophy and photoautotrophy by also showing a 15% increase in the oxygen yields previously observed in a heterotrophic process. The oxygen yields along with the fact that inorganic carbon was not supplemented in the culture suggest the process helps reduce the mass transfer requirements of the mixotrophic culture by maintaining the balance between photoautotrophic and heterotrophic metabolisms.

The system operated in nitrogen-sufficient/carbon-limiting conditions (non-detectable residual glucose) in order maximize phycocyanin production, which was 2.5% pure phycocyanin per dry biomass weight during steady state operation. The pH was set at 2.5 which prevented other contaminates from competing with the acidophilic mixotroph. The cultures were maintained clean during the 2-week continuous operation.

This method of using carbon to nitrogen ration in a culture/culture medium (feedstock) thus represents another aspect of the invention that can be applied to the culture of different microalgae species. The process was then repeated using different carbon to nitrogen ratios in the feedstock. The results confirmed that carbon to nitrogen ratio could be used to control the substrate yields (Tables 5) and therefore to balance mixotrophic growth with the relative contribution from each metabolism.

TABLE 5
Impact on the C:N ratio in the continuous feed on the Galdieria
sulphuraria substrate yields.

| | C:N ratio (w/w) | | |
|---|---|---|---|
| | 10 | 13 | 20 |
| Cell density | 5 | 5.1 | 5.2 |
| Glucose yields | 1 | 0.5 | 0.3 |
| Productivity (g/ld) | 1 | 1.5 | 1 |

In one non-limiting embodiment, a method of controlling availability of light during mixotrophic microalgae culturing while maintaining productivity may comprise: providing a culture of microalgae in a culturing vessel at a predetermined culture density of a first dry weight pre volume allowing a first availability of light within the culture of microalgae and a daily biomass productivity for the microorganisms; supplying light to the microalgae culture; supplying fresh media comprising organic carbon to the microalgae culture continuously through a pH auxostat system; and removing a portion of the microalgae culture from the culturing vessel continuously, wherein the culture density of the microalgae culture in the culture vessel stays substantially constant or decreases to a dry weight per volume less than the firs dry weight per volume over time with a corresponding availability of light greater than or equal to the first availability of light, and the daily biomass productivity remains substantially constant over time. In some embodiments, the supplied light may comprise constant lighting of the same intensity and quantity or a consistent schedule of supplying light. In some embodiments, the microalgae is Chlorella.

In another non-limiting embodiment, a method of selecting for a population of highest productivity cells in a microalgae culture during the culturing process may comprise: providing a culture of microalgae in a culturing vessel at a predetermined culture density of a first dry weight per volume with a daily biomass productivity; supplying fresh media comprising organic carbon to the microalgae culture continuously through a pH auxostat system; and removing a portion of the microalgae culture from the culturing vessel continuously, wherein the culture density of the microalgae culture in the culturing vessel decreases to at least one second dry weight per volume less than the first dry weight per volume while the daily biomass productivity remains substantially constant over time to produce a microalgae culture of a population of highest biomass producing cells in the culturing vessel. In some embodiments, the method may further comprise supplying light to the microalgae culture.

In another non-limiting embodiment, a method of increasing productivity in a microalgae culture may comprise: culturing a population of microalgae in a culturing vessel at a first culture density measured by a first dry weight per volume with a discrete supply of organic carbon to achieve a predetermined second culture density measured by a second dry weight per volume which is larger than the first culture density; supplying fresh media comprising organic carbon to the microalgae culture at the second culture density continuously through a pH auxostat system when the culture density is maintained substantially constant at the second dry weight per volume; and removing a portion of the microalgae culture from the culturing vessel continuously to maintain the second culture density as substantially constant, wherein the microalgae culture produces more over a given time period of at least one selected from the group consisting of biomass, protein, lipids, pigments, carbohydrates, and phytohormones than a reference culture not continuously receiving fresh media and continuously removing a portion of the microalgae culture. In some embodiments, the method may further comprise supplying light to the microalgae culture. In some embodiments, the culturing vessel may be opened or closed.

In another non-limiting embodiment, a method of increasing longevity of a microalgae culture may comprise: culturing a population of microalgae in a culturing vessel at a first culture density measured by a first dry weight per volume with a discrete supply of organic carbon to achieve a predetermined second culture density measured by a second dry weight per volume which is larger than the first culture density; supplying fresh media comprising organic carbon to the microalgae culture at the second culture density continuously through a pH auxostat system when the culture density is maintained substantially constant at the second dry weight per volume; and removing a portion of the microalgae culture from the culturing vessel continuously to maintain the second culture density as substantially constant, wherein the microalgae culture sustains microalgae cell growth longer than a reference culture not continuously receiving fresh media and continuously removing a portion of the microalgae culture. In some embodiments, the method may further comprise supplying light to the microalgae culture.

In another non-limiting embodiment, a method for producing microalgae may comprise, culturing a population of microalgae in a closed first culturing vessel at a substantially constant culture density measured by a dry weight per volume; supplying a fresh media comprising organic carbon to the microalgae culture continuously through a pH auxostat system; removing a portion of the microalgae culture from the closed first culturing vessel to a harvesting vessel continuously to maintain the culture density of the microalgae culture in the closed first culturing vessel as substantially constant; and transferring the at least some of the microalgae in harvesting vessel to a second culturing vessel which does not supply fresh media comprising organic carbon to the microalgae culture continuously through a pH auxostat system and does not remove a portion of the microalgae culture form the second culturing vessel continuously to maintain the culture density of the microalgae culture in the second culturing vessel as substantially constant.

In another non-limiting embodiment, a method for producing microalgae may comprise: culturing a population of microalgae in a first culturing vessel in a batch culturing mode; transferring the population of microalgae from the first culturing vessel to a second culturing vessel; supplying fresh media comprising organic carbon to the microalgae culture in the second culturing vessel continuously through a pH auxostat system; and removing a portion of the microalgae culture from the second culturing vessel to a harvesting vessel continuously to maintain the culture density of the microalgae culture in the second culturing vessel as substantially constant.

In another non-limiting embodiment, a system for continuous culturing of microalgae may comprise: a culturing vessel configured to culture microalgae in an aqueous culture medium; a pH auxostat system configured to provide fresh media to the culturing vessel; at least one harvesting vessel configured to receive at least a portion of the microalgae culture, and provide agitation and carbon dioxide to the microalgae culture and/or the at least the portion of the microalgae culture; and harvesting means configured to transfer at least a portion of the microalgae culture from the culture vessel to the at least one harvesting vessel by gravity. In some embodiments, the culturing vessel may be open. In some embodiments, the culturing vessel may be closed.

In another non-limiting embodiment, a method of controlling light availability during mixotrophic microalgae culturing while maintaining productivity, the method comprising: providing a microalgae culture in a culturing vessel at a first culture density having a first dry weight per volume and with a daily biomass productivity of the microalgae culture, allowing a first light availability within the microalgae culture; supplying light to the microalgae culture; continuously supplying to the microalgae culture through a pH auxostat system fresh media comprising organic carbon; and continuously removing a portion of the microalgae culture from the culturing vessel over a period of time, allowing a second light availability within the microalgae culture, the second light availability being greater than or equal to the first light availability, and substantially constantly maintaining the daily biomass productivity of the microalgae culture over the period of time, wherein a culture density of the microalgae culture in the culturing vessel remains substantially constant at the first culture density having the first dry weight per volume or decreases to a second culture density having a second dry weight per volume less than the first dry weight per volume.

In another non-limiting embodiment, a method of culturing microalgae to maximize cell productivity during the culturing process, the method comprising: providing a microalgae culture in a culturing vessel at a first culture density having a first dry weight per volume and with a daily biomass productivity of the microalgae culture; continuously supplying to the microalgae culture through a pH auxostat system fresh media comprising organic carbon; and continuously removing a portion of the microalgae culture from the culturing vessel over a period of time, wherein a culture density of the microalgae culture in the culturing vessel decreases to at least one second culture density having a second dry weight per volume less than the first dry weight per volume, and wherein the daily biomass productivity remains substantially constant over the period of time.

In another non-limiting embodiment, a method of culturing microalgae comprising: providing a microalgae culture in a culturing vessel at a first culture density having a first dry weight per volume with a discrete supply of organic carbon to achieve a second culture density having a second dry weight per volume, the second culture density being larger than the first culture density and being predetermined; continuously supplying to the microalgae culture through a pH auxostat system fresh media comprising organic carbon while substantially constantly maintaining the microalgae culture at the second culture density; and continuously removing a portion of the microalgae culture from the culturing vessel over a period of time to substantially constantly maintain the microalgae culture at the second culture density, wherein the microalgae culture produces increasingly more over the period of time of at least one of biomass, protein, lipids, pigments, carbohydrates, or phytohormones than a reference culture not continuously receiving the fresh media and not being maintained at the second culture density.

In another non-limiting embodiment, a method for producing microalgae, the method comprising: culturing a microalgae population in a first culturing vessel in a batch culturing mode; transferring the microalgae population from the first culturing vessel to a second culturing vessel; continuously supplying to the microalgae population through a pH auxostat system fresh media comprising organic carbon while the microalgae population is in the second culturing vessel; and continuously removing a portion of the microalgae culture from the culturing vessel over a period of time to substantially constantly maintain the microalgae culture at the second culture density.

In another non-limiting embodiment, a system for continuously culturing microalgae, the system comprising: a culturing vessel configured to culture a microalgae population in an aqueous culture medium: a pH auxostat system configured to provide fresh media to the culturing vessel; at least one harvesting vessel configured to receive at least a portion of the microalgae population, and provide agitation and carbon dioxide to the microalgae population; and a harvesting means configured to transfer the at least the portion of the microalgae culture from the culturing vessel to the at least one harvesting vessel by gravity.

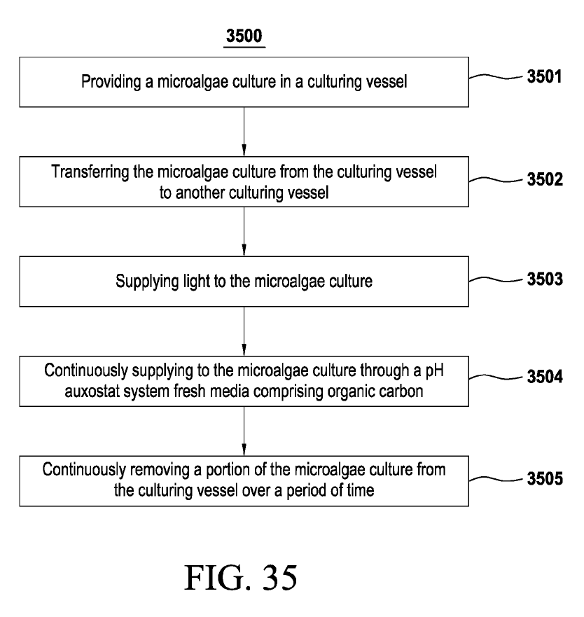

Referring now to the drawings, FIG. 35 illustrates method 3500, according to an embodiment. Method 3500 is merely exemplary and is not limited to the embodiments presented herein. Method 3500 can be employed in many different embodiments or examples not specifically depicted or described herein. In some embodiments, the activities of method 3500 can be performed in the order presented. In other embodiments, the procedures, the activities of method 3500 can be performed in any other suitable order. In still other embodiments, one or more of the activities in method 3500 can be combined or skipped.

In many embodiments, method 3500 can comprise activity 3501 of providing a microalgae culture in a culturing vessel. In some embodiments, performing activity 3501 can be similar or identical to providing a microalgae culture in a culturing vessel as described above.

In many embodiments, method 3500 can comprise activity 3502 of transferring the microalgae culture from the culturing vessel to another culturing vessel. In some embodiments, performing activity 3502 can be similar or identical to transferring the microalgae culture from the culturing vessel to another culturing vessel as described above. In some embodiments, activity 3502 can be omitted.

In many embodiments, method 3500 can comprise activity 3503 of supplying light to the microalgae culture. In some embodiments, performing activity 3503 can be similar or identical to supplying light to the microalgae culture as described above.

In many embodiments, method 3500 can comprise activity 3504 of continuously supplying to the microalgae culture through a pH auxostat system fresh media comprising organic carbon. In some embodiments, performing activity 3504 can be similar or identical to continuously supplying to the microalgae culture through a pH auxostat system fresh media comprising organic carbon as described above.

In many embodiments, method 3500 can comprise activity 3505 of continuously removing a portion of the microalgae culture from the culturing vessel over a period of time. In some embodiments, performing activity 3505 can be similar or identical to continuously removing a portion of the microalgae culture from the culturing vessel over a period of time as described above.

All references, including publications, patent applications, and patents, cited herein are hereby incorporated by reference in their entirety and to the same extent as if each reference were individually and specifically indicated to be incorporated by reference and were set forth in its entirety herein (to the maximum extent permitted by law), regardless of any separately provided incorporation of particular documents made elsewhere herein.

The use of the terms “a” and “an” and “the” and similar referents in the context of describing the invention are to be construed to cover both the singular and the plural, unless otherwise indicated herein or clearly contradicted by context.

Unless otherwise stated, all exact values provided herein are representative of corresponding approximate values (e.g., all exact exemplary values provided with respect to a particular factor or measurement can be considered to also provide a corresponding approximate measurement, modified by “about,” where appropriate). All provided ranges of values are intended to include the end points of the ranges, as well as values between the end points.

The description herein of any aspect or embodiment of the invention using terms such as “comprising”, “having,” “including,” or “containing” with reference to an element or elements is intended to provide support for a similar aspect or embodiment of the invention that “consists of”, “consists essentially of”, or “substantially comprises” that particular element or elements, unless otherwise stated or clearly contradicted by context (e.g., a composition described herein as comprising a particular element should be understood as also describing a composition consisting of that element, unless otherwise stated or clearly contradicted by context).

All headings and sub-headings are used herein for convenience only and should not be construed as limiting the invention in any way.

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

The citation and incorporation of patent documents herein is done for convenience only and does not reflect any view of the validity, patentability, and/or enforceability of such patent documents.

This invention includes all modifications and equivalents of the subject matter recited in the claims and/or aspects appended hereto as permitted by applicable law.

1. A method of controlling light availability during mixotrophic microalgae culturing while maintaining productivity, the method comprising:
providing a microalgae culture in a culturing vessel at a first culture density having a first dry weight per volume and with a daily biomass productivity of the microalgae culture, allowing a first light availability within the microalgae culture;
supplying light to the microalgae culture;
continuously supplying to the microalgae culture through a pH auxostat system fresh media comprising organic carbon; and
continuously removing a portion of the microalgae culture from the culturing vessel over a period of time, allowing a second light availability within the microalgae culture, the second light availability being greater than or equal to the first light availability, and substantially constantly maintaining the daily biomass productivity of the microalgae culture over the period of time, wherein a culture density of the microalgae culture in the culturing vessel remains substantially constant at the first culture density having the first dry weight per volume or decreases to a second culture density having a second dry weight per volume less than the first dry weight per volume.

2. The method of claim 1, wherein:
the culturing vessel is closed.

3. The method of claim 1, wherein:
the culturing vessel is open.

4. The method of any one of claim 1, wherein:
supplying the light to the microalgae culture comprises:
supplying the light to the microalgae culture with a constant intensity and a constant quantity.

5. The method of any one of claim 1, wherein:
supplying the light to the microalgae culture comprises:
supplying the light to the microalgae culture with a consistent schedule.

6. The method of any one of claim 1, wherein:
the microalgae culture comprises Chlorella.

7. A method of culturing microalgae to maximize cell productivity during the culturing process, the method comprising:
providing a microalgae culture in a culturing vessel at a first culture density having a first dry weight per volume and with a daily biomass productivity of the microalgae culture; continuously supplying to the microalgae culture through a pH auxostat system fresh media comprising organic carbon; and
continuously removing a portion of the microalgae culture from the culturing vessel over a period of time, wherein a culture density of the microalgae culture in the culturing vessel decreases to at least one second culture density having a second dry weight per volume less than the first dry weight per volume, and wherein the daily biomass productivity remains substantially constant over the period of time.

8. The method of claim 1, wherein:
the culturing vessel is closed.

9. The method of claim 1, wherein:
the culturing vessel is open.

10. The method of any one of claim 7, further comprising:
supplying light to the microalgae culture;
wherein:
supplying the light to the microalgae culture comprises at least one of:
supplying the light to the microalgae culture with a constant intensity and a constant quantity; or
supplying the light to the microalgae culture with a consistent schedule.

11. The method of any one of claim 7, wherein:
the microalgae culture comprises Chlorella.

12. A method of culturing microalgae comprising:
providing a microalgae culture in a culturing vessel at a first culture density having a first dry weight per volume with a discrete supply of organic carbon to achieve a second culture density having a second dry weight per volume, the second culture density being larger than the first culture density and being predetermined;
continuously supplying to the microalgae culture through a pH auxostat system fresh media comprising organic carbon while substantially constantly maintaining the microalgae culture at the second culture density; and
continuously removing a portion of the microalgae culture from the culturing vessel over a period of time to substantially constantly maintain the microalgae culture at the second culture density, wherein the microalgae culture produces increasingly more over the period of time of at least one of biomass, protein, lipids, pigments, carbohydrates, or phytohormones than a reference culture not continuously receiving the fresh media and not being maintained at the second culture density.

13. The method of claim 12, wherein:
the culturing vessel is closed.

14. The method of claim 12, wherein:
the culturing vessel is open.

15. The method of any one of claim 12, further comprising:
supplying light to the microalgae culture;
wherein:
supplying the light to the microalgae culture comprises at least one of:
supplying the light to the microalgae culture with a constant intensity and a constant quantity; or
supplying the light to the microalgae culture with a consistent schedule.

16. A method for producing microalgae, the method comprising:
culturing a microalgae population in a first culturing vessel in a batch culturing mode; transferring the microalgae population from the first culturing vessel to a second culturing vessel;
continuously supplying to the microalgae population through a pH auxostat system fresh media comprising organic carbon while the microalgae population is in the second culturing vessel; and
continuously removing a portion of the microalgae culture from the culturing vessel over a period of time to substantially constantly maintain the microalgae culture at the second culture density.

17. The method of claim 16, further comprising:
supplying light to the microalgae population;
wherein:
supplying the light to the microalgae population comprises at least one of:
supplying the light to the microalgae population with a constant intensity and a constant quantity; or
supplying the light to the microalgae population with a consistent schedule.

18. A system for continuously culturing microalgae, the system comprising:
a culturing vessel configured to culture a microalgae population in an aqueous culture medium;
a pH auxostat system configured to provide fresh media to the culturing vessel;
at least one harvesting vessel configured to receive at least a portion of the microalgae population, and provide agitation and carbon dioxide to the microalgae population; and
a harvesting means configured to transfer the at least the portion of the microalgae culture from the culturing vessel to the at least one harvesting vessel by gravity.

19. The system of claim 18, wherein:
the culturing vessel is closed.

20. The system of claim 18, wherein:
the culturing vessel is open.
